(19) United States
(12) Patent Application Publication
(10) Pub. No.: US 2023/0141271 A1
(43) Pub. Date: May 11, 2023

(54) OLIGOMER AND COMPOSITION

(71) Applicant: INDUSTRIAL TECHNOLOGY RESEARCH INSTITUTE, Hsinchu (TW)

(72) Inventors: Cheng-Po KUO, Taipei City (TW); Kuan-Han WU, Taipei City (TW); Shin-Liang KUO, Hsinchu City (TW); Shu-Chuan HUANG, Zhubei City (TW); Wen-Sheng CHANG, Miaoli County (TW); Jian-Yi HANG, Zhonghe City (TW)

(73) Assignee: INDUSTRIAL TECHNOLOGY RESEARCH INSTITUTE, Hsinchu (TW)

(21) Appl. No.: 17/940,451

(22) Filed: Sep. 8, 2022

Related U.S. Application Data
(60) Provisional application No. 63/294,002, filed on Dec. 27, 2021.

(30) Foreign Application Priority Data
Nov. 5, 2021 (TW) 110141253
Aug. 16, 2022 (TW) 111130711

(51) Int. Cl. C08G 73/10; C07D 519/00

(52) U.S. Cl. CPC C08G 73/1046; C07D 519/00

(57) ABSTRACT

An oligomer includes a repeating unit

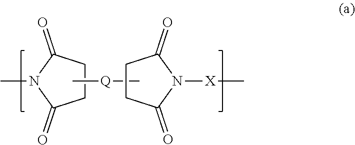

and a repeating unit

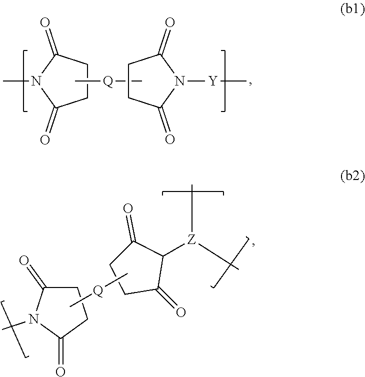

or a combination thereof. The repeating number of the repeating unit (a) and the repeating number of the repeating unit (b1), (b2), or a combination thereof have a ratio of 1:1 to 20:1. Each Q is independently substituted or unsubstituted aliphatic group, cycloaliphatic group, aromatic group, or siloxane group. X can be

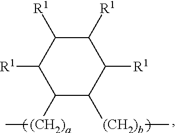

and Y can be

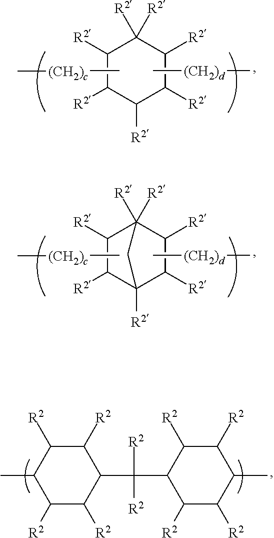

or a combination thereof, and Z can be

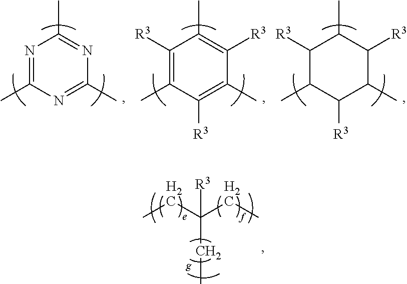

or a combination thereof.

### CROSS REFERENCE TO RELATED APPLICATIONS

This application claims the benefit of U.S. Provisional Application No. 63/294,002 filed on Dec. 27, 2021, the entirety of which is incorporated by reference.

The present application is based on, and claims priority from, Taiwan Application Serial Number 111130711, filed on Aug. 16, 2022, which claims priority from, Taiwan Application Serial Number 110141253, filed on Nov. 5, 2021, the disclosure of which are hereby incorporated by reference herein in its entirety.

### TECHNICAL FIELD

The technical field relates to an oligomer, and in particular it relates to types and ratios of repeating units in the oligomer.

### BACKGROUND

The current communications and electronic industries are developing rapidly towards high-speed, high-frequency and high-density devices. Therefore, a polymer or oligomer having high thermal resistance, low dielectric properties, low moisture absorption, and a tough structure is the major developmental direction of current electronic packaging and high-frequency substrate materials. Polyimide is usually applied in electronic packaging and high-frequency substrate materials. However, conventional thermal curable compositions containing polyimide or imide oligomer often have problems such as poor thermal resistance. As such, designing and developing an imide oligomer with excellent dielectric properties and thermal resistance is called for.

### SUMMARY

One embodiment of the disclosure provides an oligomer, including: a repeating unit

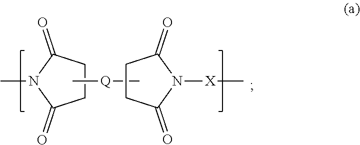

and a repeating unit

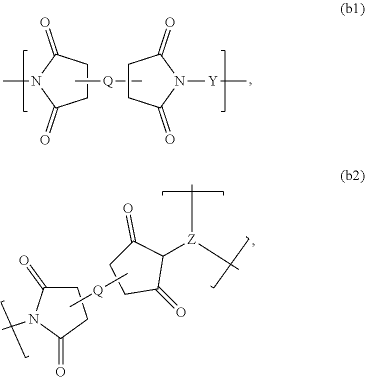

or a combination thereof, wherein the repeating number of the repeating unit (a) and the repeating number of the repeating unit (b1), (b2), or a combination thereof have a ratio of 1:1 to 20:1; each Q is independently substituted or unsubstituted aliphatic group, cycloaliphatic group, aromatic group, or siloxane group; X is

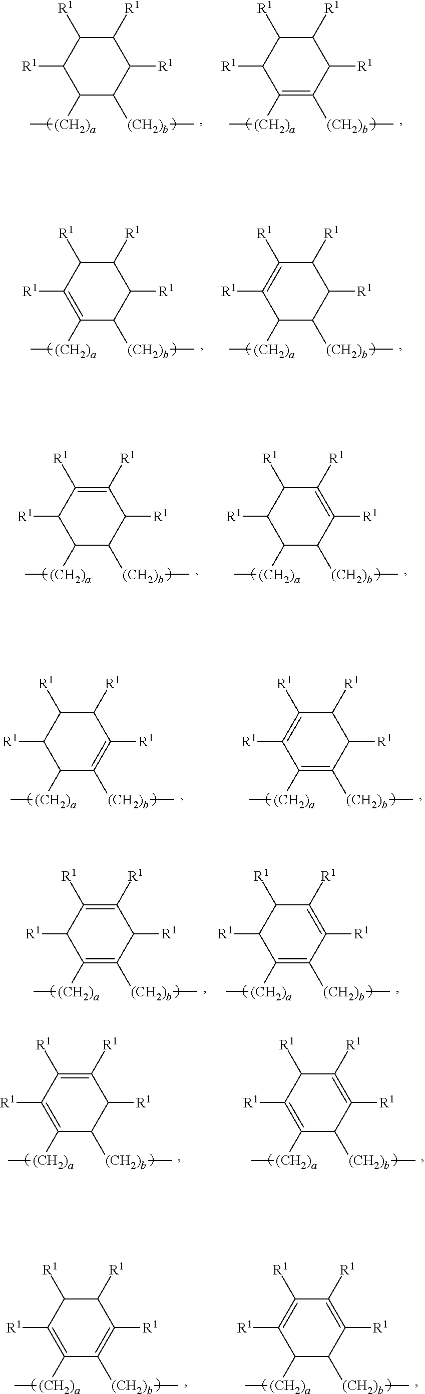

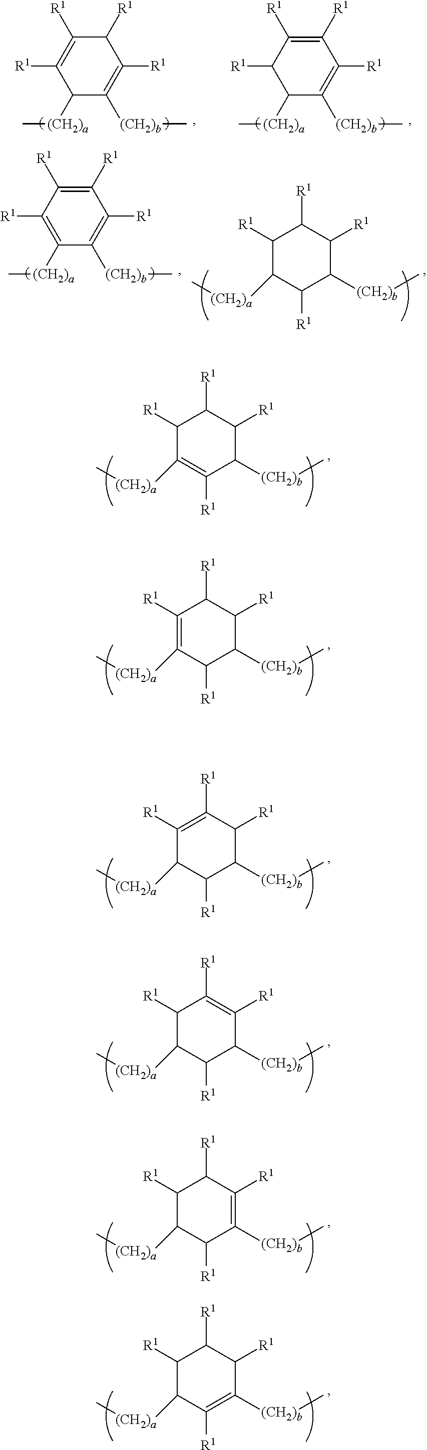

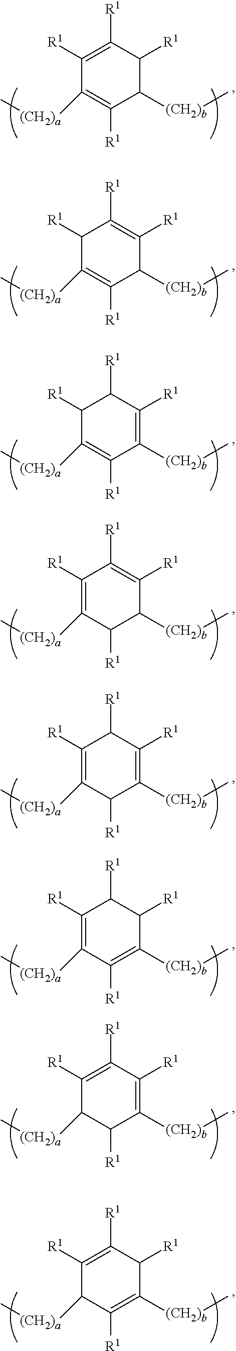

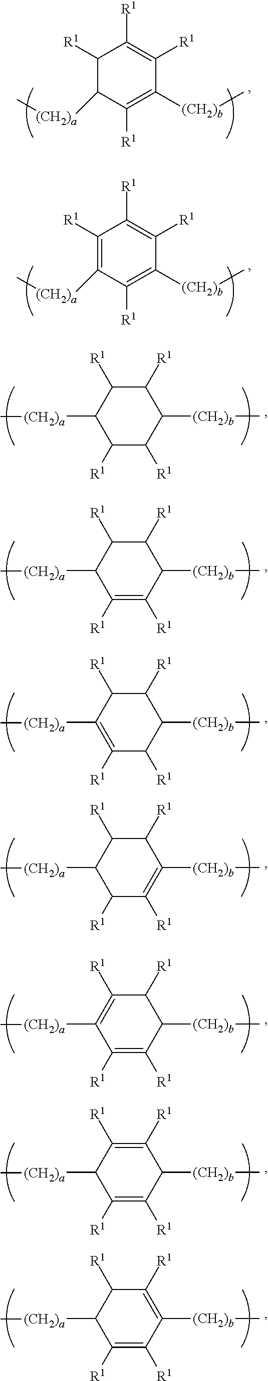

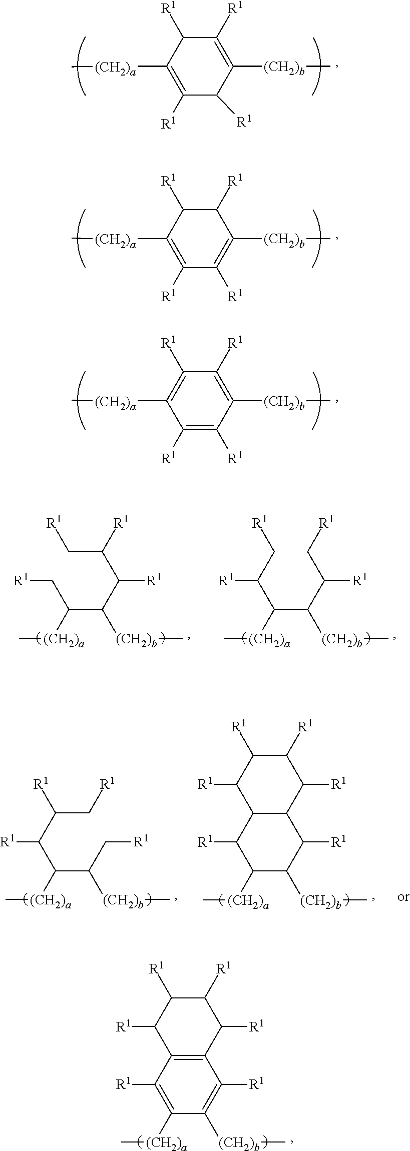

wherein 12≥a≥4; 12≥b≥4; each of R¹ is independently hydrogen, C₄₋₁₂ alkyl group, C₄₋₁₂ alkenyl group, or C₄₋₁₂ alkynyl group, and at least two R¹ is not hydrogen; Y is

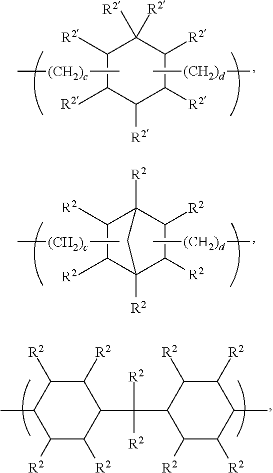

or a combination thereof, wherein c is 0 or 1; d is 0 or 1; each of R² is independently hydrogen or C₁₋₁₀ alkyl group, and at least one of R^{2′} is not hydrogen; and each of R² is independently hydrogen or C₁₋₁₀ alkyl group, and Z is

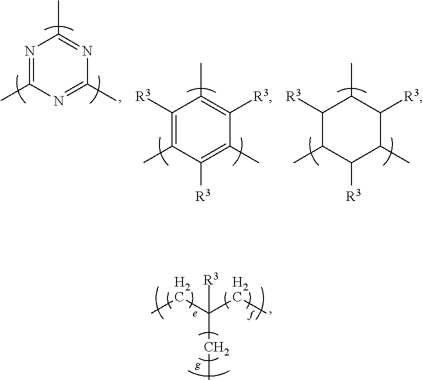

or a combination thereof, wherein 12≥e≥1; 12≥f≥1; 12≥g≥1; and each of R³ is independently hydrogen or C₁₋₁₀ alkyl group.

One embodiment of the disclosure provides a composition, including: an oligomer; and a radical initiator, wherein the oligomer has a terminal group modified to contain a double bond, and includes: a repeating unit

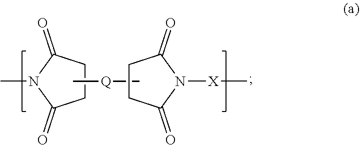

and a repeating unit

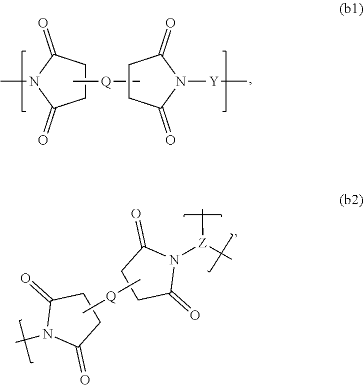

or a combination thereof, wherein the repeating number of the repeating unit (a) and the repeating number of the repeating unit (b1), (b2), or a combination thereof have a ratio of 1:1 to 20:1; each Q is independently substituted or unsubstituted aliphatic group, cycloaliphatic group, aromatic group, or siloxane group; X is

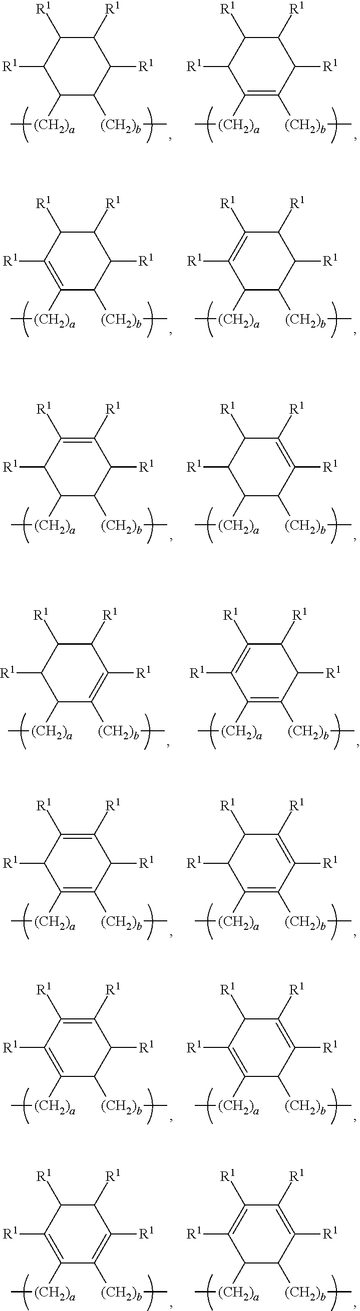

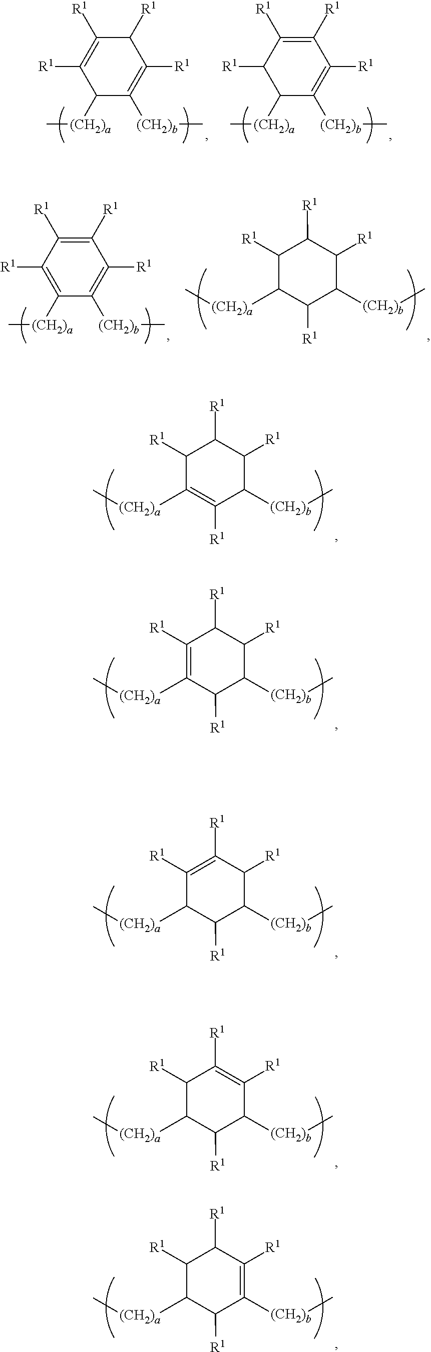

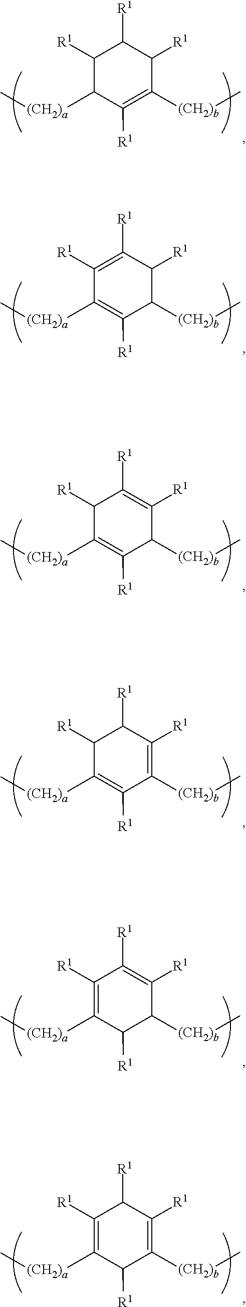

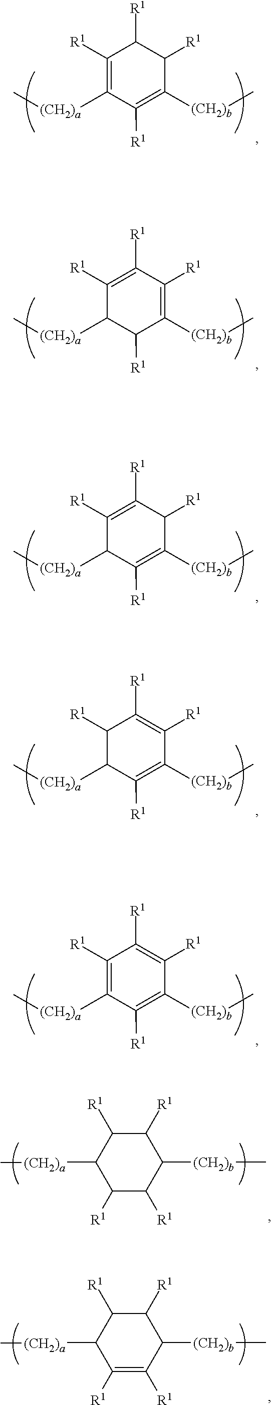

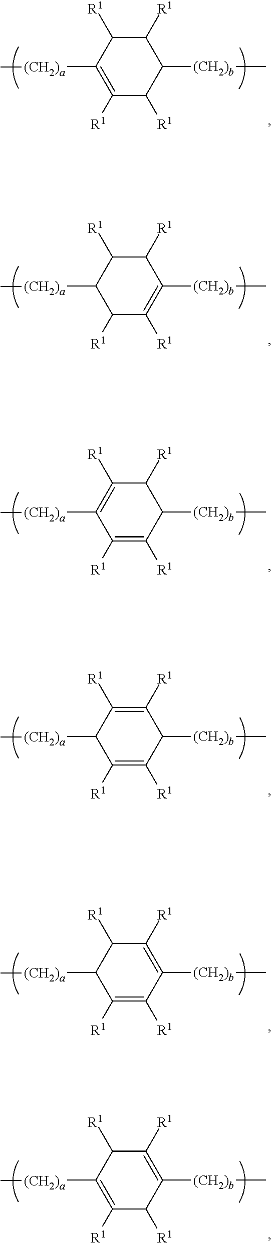

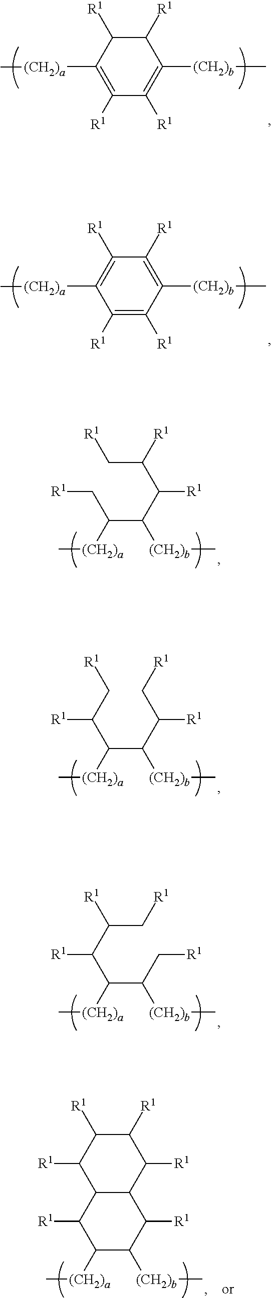

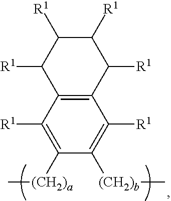

wherein 12≥a≥4; 12≥b≥4; each of R¹ is independently hydrogen, C₄₋₁₂ alkyl group, C₄₋₁₂ alkenyl group, or C₄₋₁₂ alkynyl group, and at least two R¹ is not hydrogen; Y is

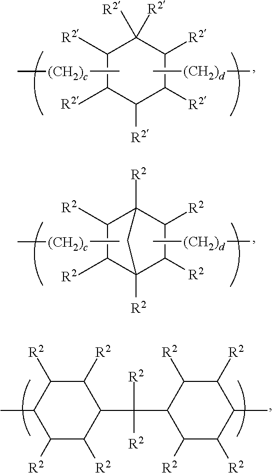

or a combination thereof, wherein c is 0 or 1; d is 0 or 1; each of R^{2′} is independently hydrogen or C₁₋₁₀ alkyl group, and at least one of R^{2′} is not hydrogen; and each of R² is independently hydrogen or C₁₋₁₀ alkyl group; and Z is

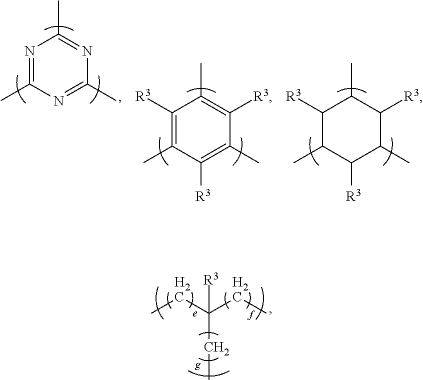

or a combination thereof, wherein 12≥e≥1; 12≥f≥1; 12≥g≥1; and each of R³ is independently hydrogen or C₁₋₁₀ alkyl group.

A detailed description is given in the following embodiments.

### DETAILED DESCRIPTION

In the following detailed description, for the purposes of explanation, numerous specific details are set forth in order to provide a thorough understanding of the disclosed embodiments. It will be apparent, however, that one or more embodiments may be practiced without these specific details.

One embodiment of the disclosure provides an oligomer, including: a repeating unit

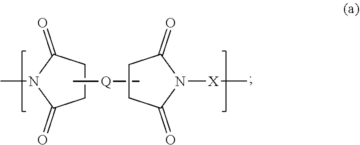

and a repeating unit

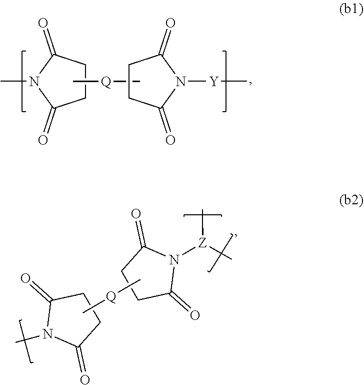

or a combination thereof. In some embodiments, the repeating number of the repeating unit (a) and the repeating number of the repeating unit (b1), (b2), or a combination thereof have a ratio of 1:1 to 20:1. If the amount of repeating unit (a) is too low, the electrical properties of the oligomer may be poor (e.g. the dielectric loss at high frequency of the composition will be too high). If the amount of repeating unit (b1), (b2), or a combination thereof is too low, the thermal stability of the oligomer may be insufficient (e.g. the glass transition temperature of the composition will be too low). In some embodiments, the repeating number of the repeating unit (a) and the repeating number of the repeating unit (b1) have a ratio of 1:1 to 5:1.

In some embodiments, the repeating units (b1) and (b2) are simultaneously used, and the repeating number of the repeating unit (b1) and the repeating number of the repeating unit (b2) have a ratio of 1:1 to 5:1. If the amount of repeating unit (b1) is too high, the electrical properties of the oligomer may be poor (e.g. the dielectric loss at high frequency of the composition will be too high). If the amount of the repeating unit (b2) is too high, the solubility of the oligomer in the solvent may be poor.

In some embodiments, each of the repeating unit (a), (b1) and (b2) independently has a repeating number of 1 to 20.

In some embodiments, each Q is independently substituted or unsubstituted aliphatic group, cycloaliphatic group, aromatic group, or siloxane group. For Example,

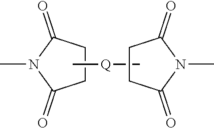

can be

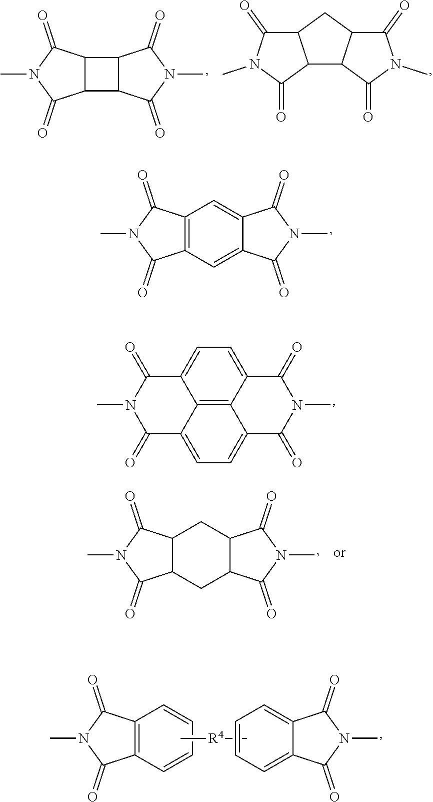

wherein R⁴ is C₁₋₅ alkylene group, —SO₂—, —O—, —C(═O)—, or a single bond. It should be understood that

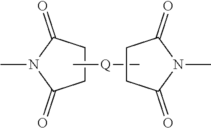

comes from

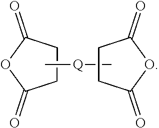

### For Example,

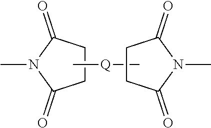

can be

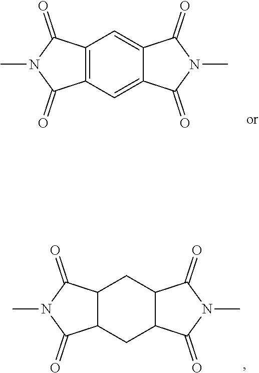

which may come from

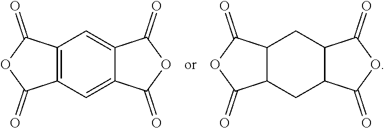 [SMILES: O=c1oc(=O)c2cc3c(=O)oc(=O)c3cc12 ; O=C1OC(=O)C2CC3C(=O)OC(=O)C3CC12]

In some embodiments, X is

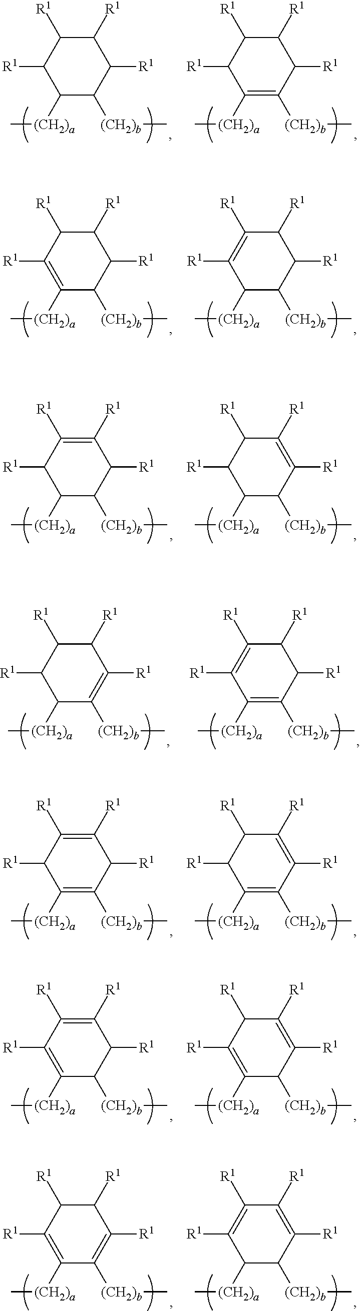

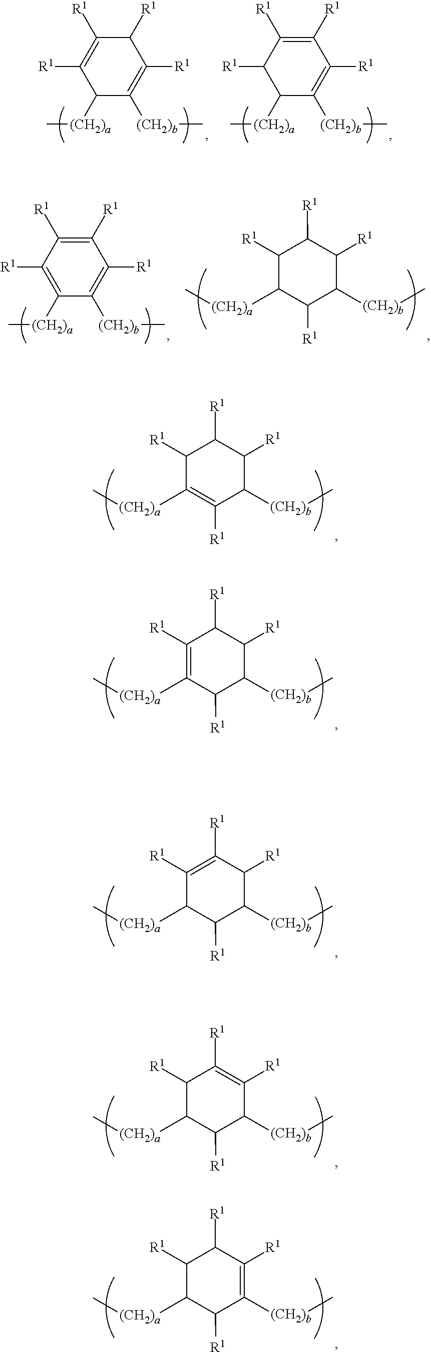

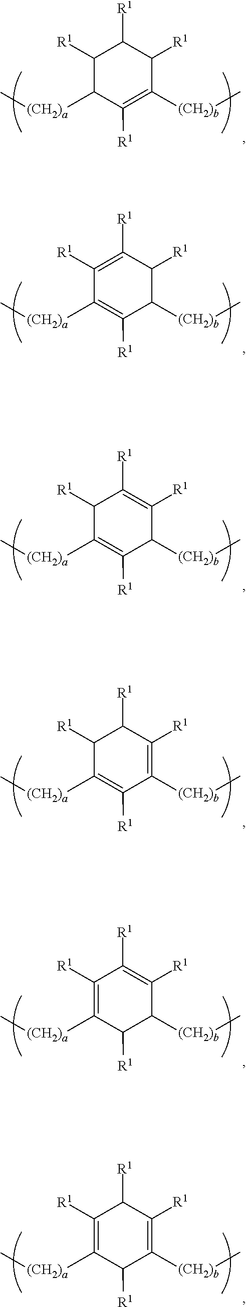

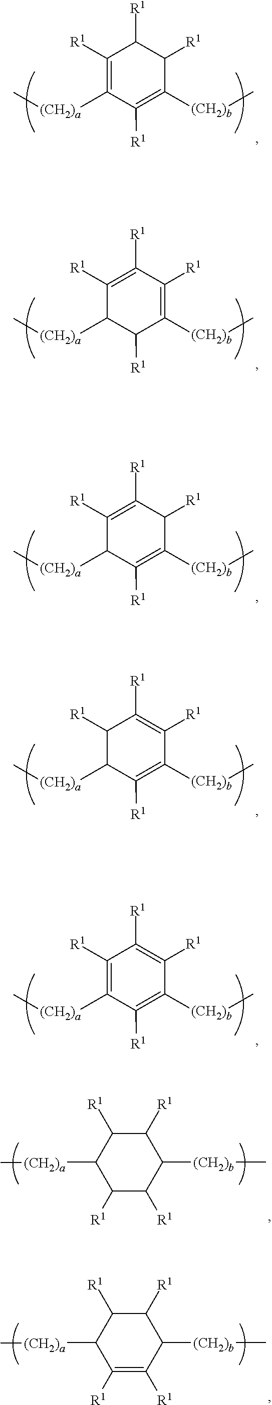

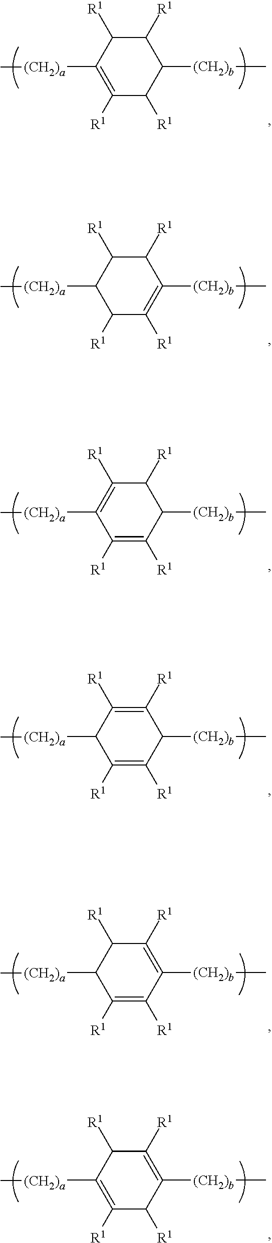

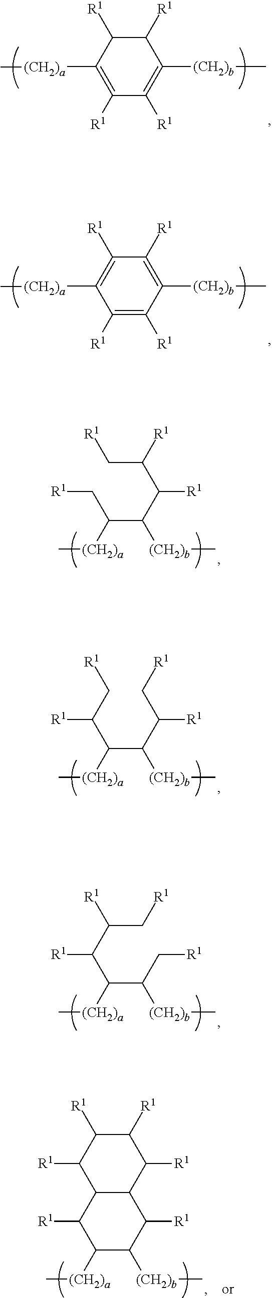

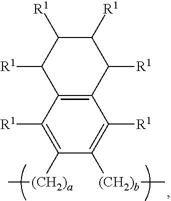

wherein 12≥a≥4; 12≥b≥4; each of R¹ is independently hydrogen, C₄₋₁₂ alkyl group, C₄₋₁₂ alkenyl group, or C₄₋₁₂ alkynyl group, and at least two R¹ is not hydrogen. In some embodiments, X comes from the commercially available dimer diamine. For example, X can be

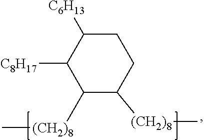

which may come from dimer diamine such as

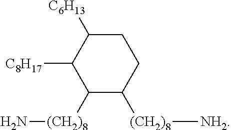

In some embodiments, Y is

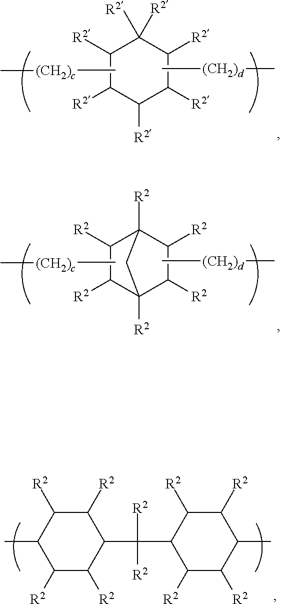

or a combination thereof, wherein c is 0 or 1; d is 0 or 1; each of R^{2′} is independently hydrogen or C₁₋₁₀ alkyl group, and at least one of R^{2′} is not hydrogen; and each of R² is independently hydrogen or C₁₋₁₀ alkyl group. For example, Y comes from diamine. For example, Y can be

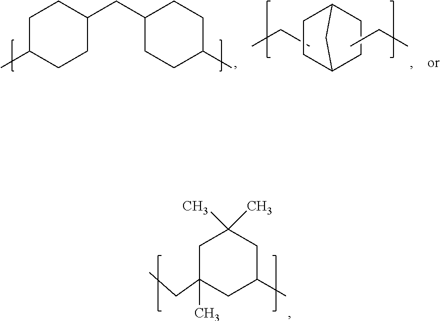

which may come from

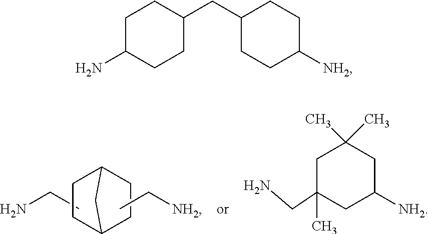

In some embodiments, Z is

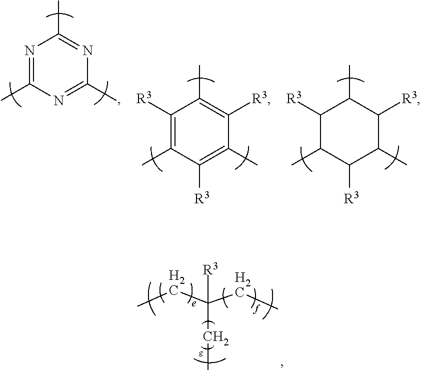

or a combination thereof, wherein 12≥e≥1; 12≥f≥1; 12≥g≥1; and each of R³ is independently hydrogen or C₁₋₁₀ alkyl group. For example, Z comes from triamine. For example, Z can be

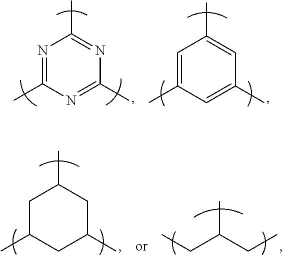

which may come from

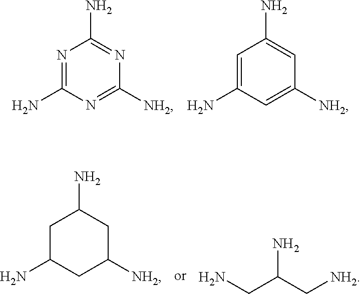 [SMILES: Nc1nc(N)nc(N)n1 ; Nc1cc(N)cc(N)c1 ; NC1CC(N)CC(N)C1 ; NCC(N)CN]

In some embodiments, the repeating unit (a) is

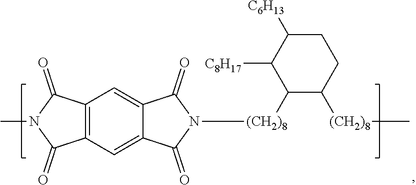

and the repeating unit (b1) is

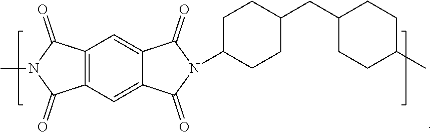

In some embodiments, the repeating unit (a) is

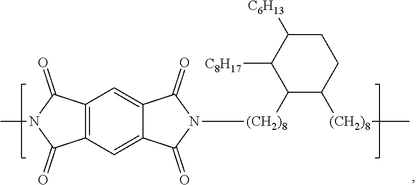

and the repeating unit (b1) is

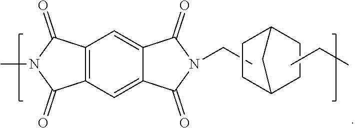

In some embodiments, the repeating unit (a) is

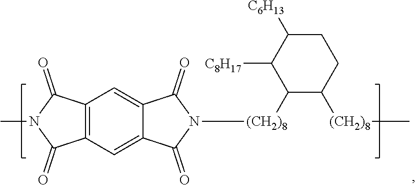

and the repeating unit (b1) is

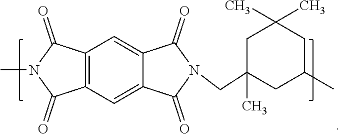

In some embodiments, the repeating unit (a) is

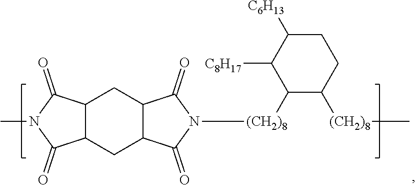

and the repeating unit (b1) is

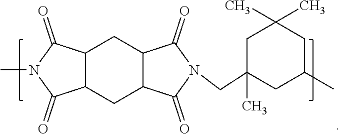

In some embodiments, the repeating unit (a) is

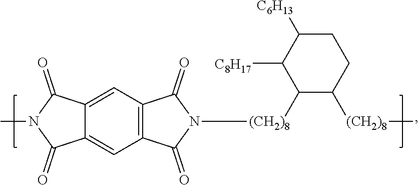

and the repeating unit (b2) is

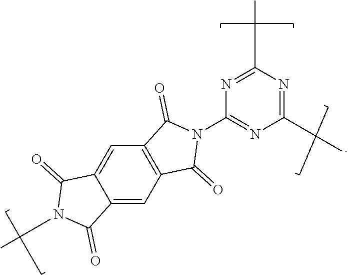

In some embodiments, the repeating unit (a) is

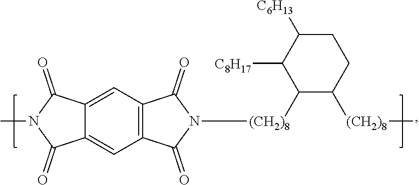

the repeating unit (b1) is

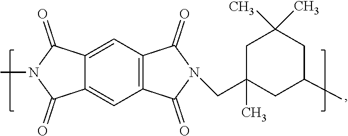

and the repeating unit (b2) is

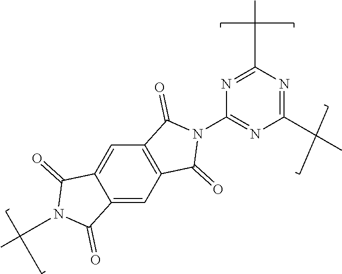

In some embodiments, the oligomer has a terminal amino group or a terminal group modified to contain a double bond. For example, bismaleic anhydride

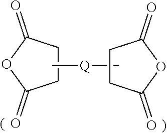

is reacted with dimer diamine (H₂N—X—NH₂) and another diamine (H₂N—Y—NH₂) and/or triamine (Z—(NH₂)₃) to form the described oligomer. The described oligomer has a terminal amino group (—NH₂). In some embodiment, the amino group of the oligomer may react with a resin containing epoxy group, isocyanate group, or another suitable functional group. Alternatively, the terminal amino group of the oligomer may react with maleic anhydride to form

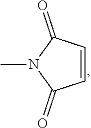

e.g. modified to a group containing a double bond.

In some embodiments, a composition may include the oligomer having a terminal group modified to contain a double bond, and a radical initiator. After applying energy to the composition, the oligomer in the composition can be radical polymerized. Alternatively, the composition may include another resin containing a double bond, and the oligomer having a terminal group modified to contain a double bond may copolymerize with the other resin containing a double bond. For example, the other resin containing a double bond can be a polyphenylene ether resin having a terminal methacryloxy group, a polyphenylene ether resin having a terminal vinyl benzyl ether group, another suitable resin, or a combination thereof. In some embodiments, the copolymer of the oligomer having a terminal group modified to contain a double bond and the modified polyphenylene ether has excellent electrical properties and thermal properties, such as low dielectric loss at high frequency and high glass transition temperature. The copolymer can be used as a high-frequency (soft or hard) substrate material, high temperature type additive, packaging material, adhesive, or the like. Note that the oligomer can be used alone or used with another resin (rather than being limited to the described modified polyphenylene ether resin).

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

In the following Examples, the compositions were tested as below: The dielectric constant (Dk) was measured according to the standard IPC-TM-650 2.5.5. The dielectric constant means the electrical insulation properties of the composition, and the lower dielectric constant means better electrical insulation. The dielectric loss (Df) was measured according to the standard IPC-TM-650 2.5.5. The dielectric loss means the ability of a material to absorbing microwaves of a certain frequency at a certain temperature. In a general communication product specification, lower dielectric loss is better. The glass transition temperature was measured by differential scanning calorimetry (DSC) according to the standard IPC-TM-650 2.4.25.

### Synthesis Example 1

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol) and 4,4′-diaminodicyclohexylmethane (3.57 g, 0.017 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (45 g). The wax product (oligomer 1) was identified by gel permeation chromatography (GPC). The oligomer 1 had a number average molecular weight of 2597 and a PDI index of 1.90. The ¹H NMR spectrum (400 MHz, CDCl₃) of the oligomer 1 is shown below: δ 8.233 (br, 2H), δ 6.654 (s, 2H), δ 6.602-6.598 (s, 2H), δ 3.721-3.684 (br, 6H), δ 3.496-3.460 (m, 3H), δ 1.668-0.835 (br, 86H). The oligomer 1 had a repeating unit (a) of

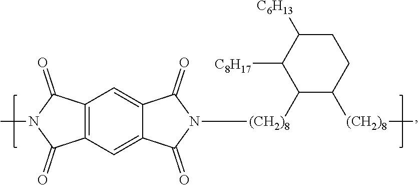

and a repeating unit (b1) of

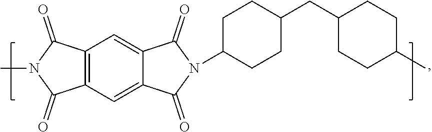

and the repeating number of the repeating unit (a) and the repeating number of the repeating unit (b1) had a ratio of 4:1. In addition, the terminal group of the oligomer 1 was

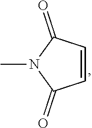

e.g. modified to contain a double bond.

### Synthesis Example 2

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (22.3 g, 0.043 mol) and 4,4′-diaminodicyclohexylmethane (9.03 g, 0.043 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (34 g). The wax product (oligomer 2) was identified by GPC. The oligomer 2 had a number average molecular weight of 1800 and a PDI index of 1.95. The ¹H NMR spectrum (400 MHz, CDCl₃) of the oligomer 2 is shown below: δ 8.235 (br, 2H), δ 6.654 (s, 2H), δ 6.608-6.592 (s, 2H), δ 3.730-3.680 (br, 6H), δ 3.500-3.456 (m, 3H), δ 1.674-0.828 (br, 86H). The oligomer 2 had a repeating unit (a) of

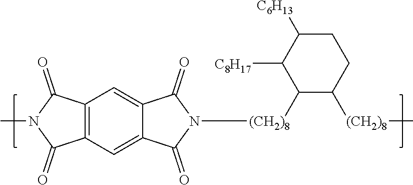

and a repeating unit (b1) of

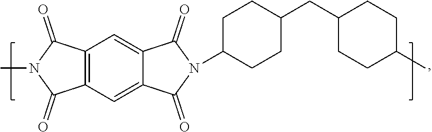

and the repeating number of the repeating unit (a) and the repeating number of the repeating unit (b1) had a ratio of 1:1. In addition, the terminal group of the oligomer 2 was

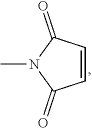

e.g. modified to contain a double bond.

### Synthesis Example 3

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (37.3 g, 0.072 mol) and 4,4′-diaminodicyclohexylmethane (2.52 g, 0.012 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (42 g). The wax product (oligomer 3) was identified by GPC. The oligomer 3 had a number average molecular weight of 3000 and a PDI index of 1.96. The ¹H NMR spectrum (400 MHz, CDCl₃) of the oligomer 3 is shown below: δ 8.233 ppm (br, 2H), δ 6.656 (s, 2H), δ 6.602-6.598 (s, 2H), δ 3.724-3.680 (br, 6H), δ 3.496-3.460 (m, 3H), δ 1.668-0.835 (br, 86H). The oligomer 3 had a repeating unit (a) of

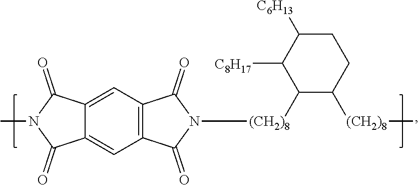

and a repeating unit (b1) of

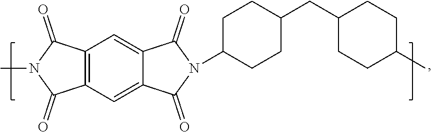

and the repeating number of the repeating unit (a) and the repeating number of the repeating unit (b1) had a ratio of 6:1. In addition, the terminal group of the oligomer 3 was

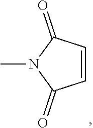

e.g. modified to contain a double bond.

### Synthesis Example 4

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol) and bis(aminomethyl)norborane (2.62 g, 0.017 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (45 g). The wax product (oligomer 4) was identified by GPC. The oligomer 4 had a number average molecular weight of 2301 and a PDI index of 1.75. The ¹H NMR spectrum (400 MHz, CDCl₃) of the oligomer 4 is shown below: δ 8.251-8.210 (br, 2H), δ 6.68-6.63 (br, 4H), δ 3.72-3.646 (br, 8H), δ 3.534-3.459 (m, 4H), δ 1.667-0.799 (br, 78H). The oligomer 4 had a repeating unit (a) of

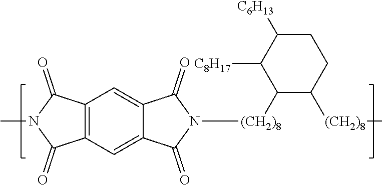

and a repeating unit (b1) of

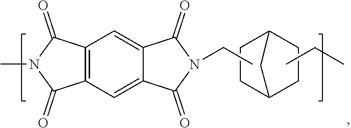

and the repeating number of the repeating unit (a) and the repeating number of the repeating unit (b1) had a ratio of 4:1. In addition, the terminal group of the oligomer 4 was

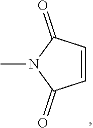

e.g. modified to contain a double bond.

### Synthesis Example 5

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (22.3 g, 0.043 mol) and bis(aminomethyl)norborane (6.63 g, 0.043 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (33 g). The wax product (oligomer 5) was identified by GPC. The oligomer 5 had a number average molecular weight of 1850 and a PDI index of 1.71. The ¹H NMR spectrum (400 MHz, CDCl₃) of the oligomer 5 is shown below: δ 8.250-8.212 (br, 2H), δ 6.70-6.62 (br, 4H), δ 3.72-3.646 (br, 8H), δ 3.536-3.458 (m, 4H), δ 1.667-0.799 (br, 78H). The oligomer 5 had a repeating unit (a) of

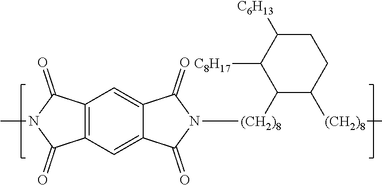

and a repeating unit (b1) of

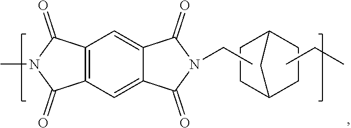

and the repeating number of the repeating unit (a) and the repeating number of the repeating unit (b1) had a ratio of 1:1. In addition, the terminal group of the oligomer 5 was

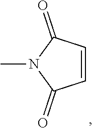

e.g. modified to contain a double bond.

### Synthesis Example 6

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (36.3 g, 0.070 mol) and bis(aminomethyl)norborane (2.18 g, 0.014 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (40 g). The wax product (oligomer 6) was identified by GPC. The oligomer 6 had a number average molecular weight of 2600 and a PDI index of 1.80. The ¹H NMR spectrum (400 MHz, CDCl₃) of the oligomer 6 is shown below: δ 8.254-8.210 (br, 2H), δ 6.66-6.61 (br, 4H), δ 3.72-3.646 (br, 8H), δ 3.536-3.460 (m, 4H), δ 1.667-0.800 (br, 78H). The oligomer 6 had a repeating unit (a) of

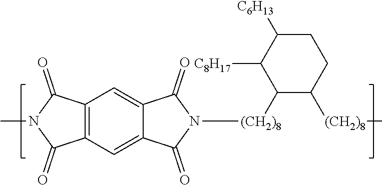

and a repeating unit (b1) of

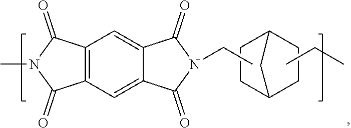

and the repeating number of the repeating unit (a) and the repeating number of the repeating unit (b1) had a ratio of 5:1. In addition, the terminal group of the oligomer 6 was

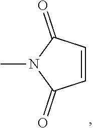

e.g. modified to contain a double bond.

### Synthesis Example 7

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol) and isophorone diamine (2.895 g, 0.017 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (45 g). The wax product (oligomer 7) was identified by GPC. The oligomer 7 had a number average molecular weight of 2631 and a PDI index of 1.67. The ¹H NMR spectrum (400 MHz, CDCl₃) of the oligomer 7 is shown below: δ 8.244-8.201 (br, 2H), δ 6.654-6.633 (s, 2H), δ 6.600 ppm (s, 2H), δ 3.720-3.683 ppm (br, 7H), δ 3.495-3.458 ppm (m, 3H), δ 1.683-0.955 ppm (br, 81H). The oligomer 7 had a repeating unit (a) of

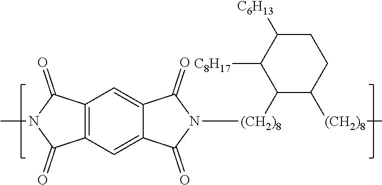

and a repeating unit (b1) of

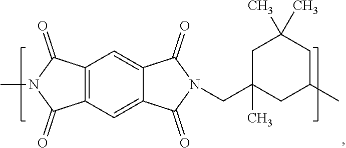

and the repeating number of the repeating unit (a) and the repeating number of the repeating unit (b1) had a ratio of 4:1. In addition, the terminal group of the oligomer 7 was

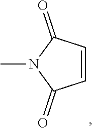

e.g. modified to contain a double bond.

### Synthesis Example 8

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (22.3 g, 0.043 mol) and isophorone diamine (7.32 g, 0.043 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (32 g). The wax product (oligomer 8) was identified by GPC. The oligomer 8 had a number average molecular weight of 2200 and a PDI index of 1.67. The ¹H NMR spectrum (400 MHz, CDCl₃) of the oligomer 8 is shown below: δ 8.246-8.200 ppm (br, 2H), δ 6.654-6.630 ppm (s, 2H), δ 6.600 ppm (s, 2H), δ 3.726-3.688 ppm (br, 7H), δ 3.495-3.458 ppm (m, 3H), δ 1.680-0.950 ppm (br, 81H). The oligomer 8 had a repeating unit (a) of

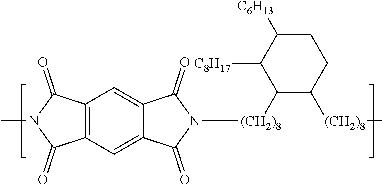

and a repeating unit (b1) of

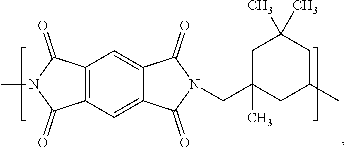

and the repeating number of the repeating unit (a) and the repeating number of the repeating unit (b1) had a ratio of 1:1. In addition, the terminal group of the oligomer 8 was

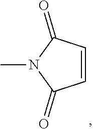

e.g. modified to contain a double bond.

### Synthesis Example 9

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (44.04 g, 0.085 mol) was then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (45 g). The wax product (oligomer 9) was identified by GPC. The oligomer 9 had a number average molecular weight of 2800 and a PDI index of 1.85. The ¹H NMR spectrum (400 MHz, CDCl₃) of the oligomer 9 is shown below: δ 8.235 ppm (br, 2H), δ 6.655 ppm (s, 2H), δ 3.723-3.686 ppm (br, 4H), δ 3.499-3.462 ppm (m, 4H), δ 1.67-0.800 ppm (br, 66H). The oligomer 9 had a repeating unit of

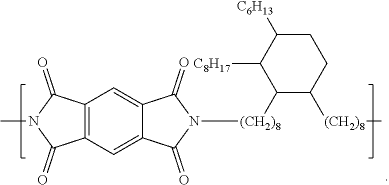

In addition, the terminal group of the oligomer 9 was

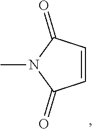

e.g. modified to contain a double bond.

### Synthesis Example 10

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (36.3 g, 0.070 mol) and isophorone diamine (2.41 g, 0.014 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (40 g). The wax product (oligomer 10) was identified by GPC. The oligomer 10 had a number average molecular weight of 2800 and a PDI index of 1.73. The ¹H NMR spectrum (400 MHz, CDCl₃) of the oligomer 10 is shown below: δ 8.242-8.200 ppm (br, 2H), δ 6.658-6.631 ppm (s, 2H), δ 6.602 ppm (s, 2H), δ 3.728-3.681 ppm (br, 7H), δ 3.490-3.458 ppm (m, 3H), δ 1.680-0.952 ppm (br, 81H). The oligomer 10 had a repeating unit (a) of

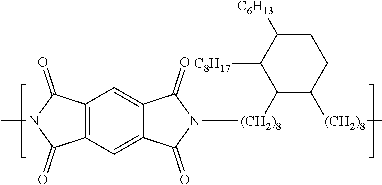

and a repeating unit (b1) of

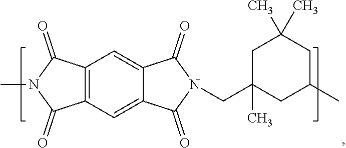

and the repeating number of the repeating unit (a) and the repeating number of the repeating unit (b1) had a ratio of 5:1. In addition, the terminal group of the oligomer 10 was

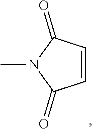

e.g. modified to contain a double bond.

### Synthesis Example 11

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (14.7 g, 0.028 mol) and isophorone diamine (9.65 g, 0.057 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (29 g). The wax product (oligomer 11) was identified by GPC. The oligomer 11 had a number average molecular weight of 2000 and a PDI index of 1.74. The ¹H NMR spectrum (400 MHz, CDCl₃) of the oligomer 11 is shown below: δ 8.238-8.200 ppm (br, 2H), δ 6.648-6.638 ppm (s, 2H), δ 6.608 ppm (s, 2H), δ 3.714-3.678 ppm (br, 7H), δ 3.490-3.452 ppm (m, 3H), δ 1.683-0.955 ppm (br, 81H). The oligomer 11 had a repeating unit (a) of

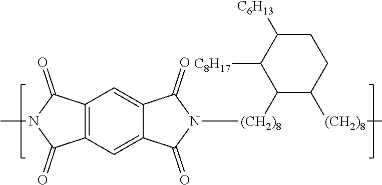

and a repeating unit (b1) of

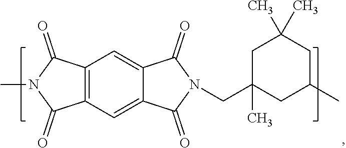

and the repeating number of the repeating unit (a) and the repeating number of the repeating unit (b1) had a ratio of 1:2. In addition, the terminal group of the oligomer 11 was

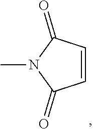

e.g. modified to contain a double bond.

### Synthesis Example 12

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (22.3 g, 0.043 mol) and isophorone diamine (7.32 g, 0.043 mol) were then added into the reactor. Subsequently, 1,2,4,5-cyclohexane tetracarboxylic dianhydride (11.2 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (33 g). The wax product (oligomer 12) was identified by GPC. The oligomer 12 had a number average molecular weight of 2150 and a PDI index of 1.80. The ¹H NMR spectrum (400 MHz, CDCl₃) of the oligomer 12 is shown below: δ 6.650-6.634 (br, 2H), δ 6.604 (s, 2H), δ 3.700-3.660 ppm (br, 7H), δ 3.496-3.460 (m, 3H), δ 2.560 (br, 4H), δ 2.064-1.800 (br, 4H), δ 1.688-0.960 ppm (br, 81H). The oligomer 12 had a repeating unit (a) of

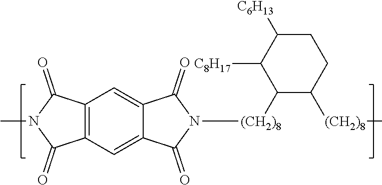

and a repeating unit (b1) of

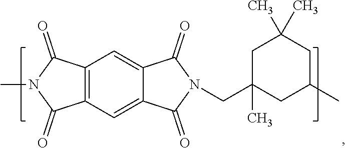

and the repeating number of the repeating unit (a) and the repeating number of the repeating unit (b1) had a ratio of 1:1. In addition, the terminal group of the oligomer 12 was

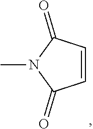

e.g. modified to contain a double bond.

### Synthesis Example 13

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (37.3 g, 0.072 mol) and isophorone diamine (2.05 g, 0.012 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (40 g). The wax product (oligomer 13) was identified by GPC. The oligomer 13 had a number average molecular weight of 2800 and a PDI index of 1.73. The ¹H NMR spectrum (400 MHz, CDCl₃) of the oligomer 13 is shown below: δ 8.246-8.200 (br, 2H), δ 6.658-6.631 (s, 2H), δ 6.606 (s, 2H), δ 3.726-3.684 (br, 7H), δ 3.490-3.458 (m, 3H), δ 1.680-0.954 (br, 81H). The oligomer 13 had a repeating unit (a) of

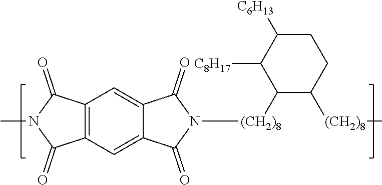

and a repeating unit (b1) of

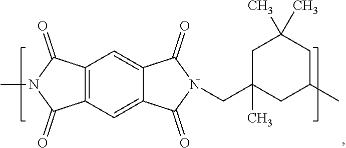

and the repeating number of the repeating unit (a) and the repeating number of the repeating unit (b1) had a ratio of 6:1. In addition, the terminal group of the oligomer 13 was

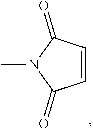

e.g. modified to contain a double bond.

### Synthesis Example 14

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (14.7 g, 0.028 mol) and isophorone diamine (9.65 g, 0.057 mol) were then added into the reactor. Subsequently, 1,2,4,5-cyclohexane tetracarboxylic dianhydride (11.2 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (29 g). The wax product (oligomer 14) was identified by GPC. The oligomer 14 had a number average molecular weight of 1800 and a PDI index of 1.78. The ¹H NMR spectrum (400 MHz, CDCl₃) of the oligomer 14 is shown below: δ 6.642-6.630 (s, 2H), δ 6.600 (s, 2H), δ 3.700-3.660 ppm (br, 7H), δ 3.498-3.458 (m, 3H), δ 2.562 (br, 4H), δ 2.062-1.800 (br, 4H), δ 1.690-0.960 ppm (br, 81H). The oligomer 14 had a repeating unit (a) of

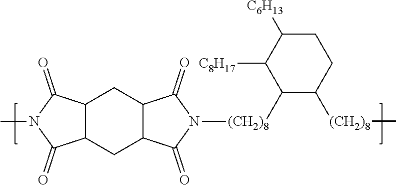

and a repeating unit (b1) of

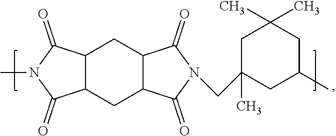

and the repeating number of the repeating unit (a) and the repeating number of the repeating unit (b1) had a ratio of 1:2. In addition, the terminal group of the oligomer 14 was

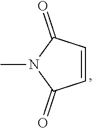

e.g. modified to contain a double bond.

### Synthesis Example 15

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol) and melamine (0.95 g, 0.0075 mol) were then added into the reactor. Subsequently, pyromellitic dianhydride (13.08 g, 0.060 mol) was slowly added into the reactor. Dean stark distillation apparatus and a condensing apparatus were connected to the reactor, and the reactants were heated to reflux until the reaction finished. The reaction was cooled to room temperature, maleic anhydride (9.8 g, 0.1 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (40 g). The wax product (oligomer 15) was identified by GPC. The oligomer 15 had a number average molecular weight of 3000 and a PDI index of 1.80. The ¹H NMR spectrum (400 MHz, CDCl₃) of the oligomer 15 is shown below: δ 8.234 (br, 2H), δ 6.655 (s, 2H), δ 3.722-3.685 ppm (br, 4H), δ 3.497-3.461 (m, 4H), δ 1.669-0.800 (br, 66H). The oligomer 15 had a repeating unit (a) of

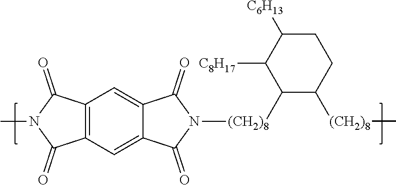

and a repeating unit (b2) of

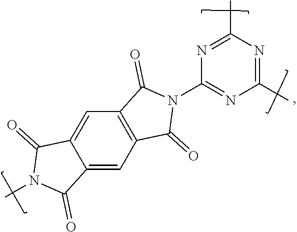

and the repeating number of the repeating unit (a) and the repeating number of the repeating unit (b2) had a ratio of 9:1. In addition, the terminal group of the oligomer 15 was

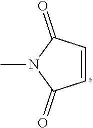

e.g. modified to contain a double bond.

### Synthesis Example 16

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (23.4 g, 0.045 mol) and melamine (1.9 g, 0.015 mol) were then added into the reactor. Subsequently, pyromellitic dianhydride (9.81 g, 0.045 mol) was slowly added into the reactor. Dean stark distillation apparatus and a condensing apparatus were connected to the reactor, and the reactants were heated to reflux until the reaction finished. The reaction was cooled to room temperature, maleic anhydride (9.8 g, 0.1 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (30 g). The wax product (oligomer 16) was identified by GPC. The oligomer 16 had a number average molecular weight of 2000 and a PDI index of 1.90. The ¹H NMR spectrum (400 MHz, CDCl₃) of the oligomer 16 is shown below: δ 8.234 (br, 2H), δ 6.655 (s, 2H), δ 3.722-3.685 ppm (br, 4H), δ 3.497-3.461 (m, 4H), δ 1.669-0.800 (br, 66H). The oligomer 16 had a repeating unit (a) of

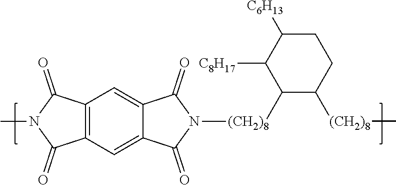

and a repeating unit (b2) of

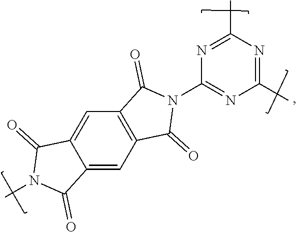

and the repeating number of the repeating unit (a) and the repeating number of the repeating unit (b2) had a ratio of 3:1. In addition, the terminal group of the oligomer 16 was

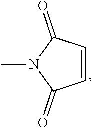

e.g. modified to contain a double bond.

### Synthesis Example 17

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol) and 1,2-diaminocyclohexane (1.90 g, 0.017 mol) were then added into the reactor. Subsequently, pyromellitic dianhydride (10.9 g, 0.05 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (41 g). The wax product (oligomer 17) was identified by GPC. The oligomer 17 had a number average molecular weight of 1800 and a PDI index of 1.81. The ¹H NMR spectrum (400 MHz, CDCl₃) of the oligomer 17 is shown below: δ 8.244-8.201 (br, 2H), δ 6.660-6.630 (s, 2H), δ 6.606 ppm (s, 2H), δ 3.724-3.680 (br, 7H), δ 3.496-3.450 (m, 3H), δ 1.683-0.955 (br, 74H). The oligomer 17 had a repeating unit (a) of

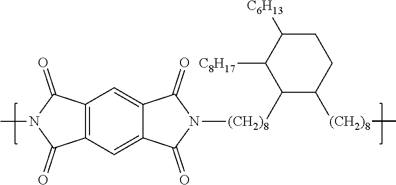

and a repeating unit (b1) of

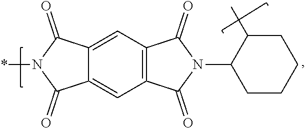

and the repeating number of the repeating unit (a) and the repeating number of the repeating unit (b1) had a ratio of 4:1. In addition, the terminal group of the oligomer 17 was

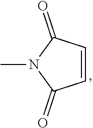

e.g. modified to contain a double bond.

### Synthesis Example 18

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (26.0 g, 0.050 mol) and 1,2-diaminocyclohexane (5.70 g, 0.050 mol) were then added into the reactor. Subsequently, pyromellitic dianhydride (13.0 g, 0.06 mol) was slowly added into the reactor. Dean stark distillation apparatus and a condensing apparatus were connected to the reactor, and the reactants were heated to reflux until the reaction finished. The reaction was cooled to room temperature, maleic anhydride (12.8 g, 0.13 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (35 g). The wax product (oligomer 18) was identified by GPC. The oligomer 18 had a number average molecular weight of 1700 and a PDI index of 1.75. The ¹H NMR spectrum (400 MHz, CDCl₃) of the oligomer 18 is shown below: δ 8.244-8.201 (br, 2H), δ 6.654-6.632 (s, 2H), δ 6.604 ppm (s, 2H), δ 3.724-3.680 (br, 7H), δ 3.496-3.452 (m, 3H), δ 1.683-0.950 (br, 74H). The oligomer 18 had a repeating unit (a) of

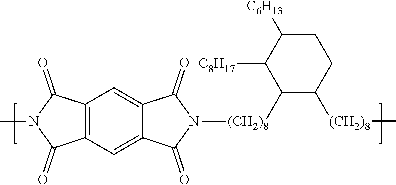

and a repeating unit (b1) of

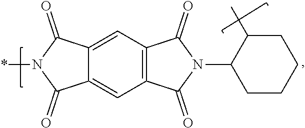

and the repeating number of the repeating unit (a) and the repeating number of the repeating unit (b1) had a ratio of 1:1. In addition, the terminal group of the oligomer 18 was

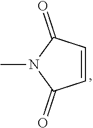

e.g. modified to contain a double bond,

### Synthesis Example 19

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol), Isophorone diamine (2.90 g, 0.017 mol), and melamine (0.95 g, 0.0075 mol) were then added into the reactor. Subsequently, pyromellitic dianhydride (15.0 g, 0.069 mol) was slowly added into the reactor. Dean stark distillation apparatus and a condensing apparatus were connected to the reactor, and the reactants were heated to reflux until the reaction finished. The reaction was cooled to room temperature, maleic anhydride (9.8 g, 0.1 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (43 g). The wax product (oligomer 19) was identified by GPC. The oligomer 19 had a number average molecular weight of 2800 and a PDI index of 1.85. The ¹H NMR spectrum (400 MHz, CDCl₃) of the oligomer 19 is shown below: δ 8.244-8.201 (br, 2H), δ 6.654-6.634 (s, 2H), δ 6.600 ppm (s, 2H), δ 3.720-3.684 (br, 7H), δ 3.496-3.458 (m, 3H), S 1.683-0.955 (br, 81H). The oligomer 19 had a repeating unit (a) of

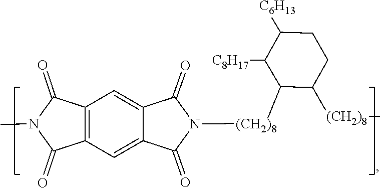

a repeating unit (b1) of

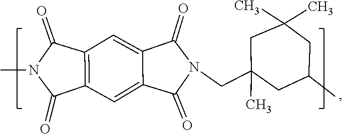

and a repeating unit (b2) of

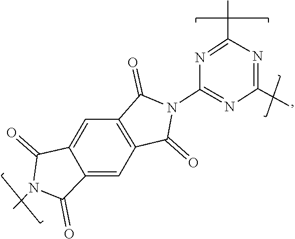

and the repeating number of the repeating unit (a), the repeating number of the repeating unit (b1), and the repeating number of the repeating unit (b2) had a ratio of 9:2:1. In addition, the terminal group of the oligomer 19 was

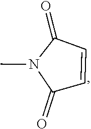

e.g. modified to contain a double bond,

### Synthesis Example 20

250 mL of toluene was added into a reactor, and triethylamine (35 g, 0.35 mol) was added into the reactor. Methanesulfonic acid (35 g, 0.35 mol) was slowly and dropwise added into the reactor to form an ammonium salt, and continuously stirred for 10 minutes. Aliphatic dimer diamine (35.2 g, 0.068 mol), Isophorone diamine (2.60 g, 0.015 mol), and melamine (1.30 g, 0.015 mol) were then added into the reactor. Subsequently, pyromellitic dianhydride (13.08 g, 0.060 mol) was slowly added into the reactor. Dean stark distillation apparatus and a condensing apparatus were connected to the reactor, and the reactants were heated to reflux until the reaction finished. The reaction was cooled to room temperature, maleic anhydride (9.8 g, 0.1 mol) and methanesulfonic acid (5 g) were then added into the reactor, and the reactants were re-heated to reflux until the reaction finished. The reaction product was cooled to room temperature, and 100 g of toluene was then added to the reactor and stood to separate layers. The reaction product was poured out, the salt was rinsed with toluene (2*100 g), and the extraction was combined and stood overnight to completely separate the toluene layer and the salt. The toluene layer was filtered by silica gel, and the solvent thereof was removed by vacuum to obtain a wax product of deep color (41 g). The wax product (oligomer 20) was identified by GPC. The oligomer 20 had a number average molecular weight of 2000 and a PDI index of 1.83. The ¹H NMR spectrum (400 MHz, CDCl₃) of the oligomer 20 is shown below: δ 8.244-8.200 (br, 2H), δ 6.654-6.633 (s, 2H), δ 6.600 ppm (s, 2H), δ 3.720-3.686 (br, 7H), δ 3.496-3.458 (m, 3H), δ 1.684-0.956 (br, 81H). The oligomer 20 had a repeating unit (a) of

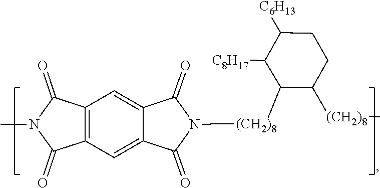

a repeating unit (b1) of

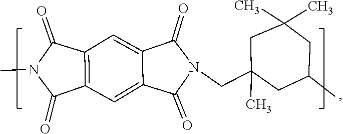

and a repeating unit (b2) of

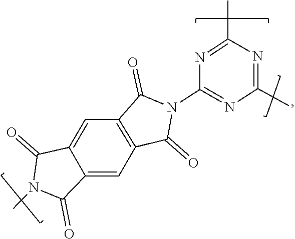

and the repeating number of the repeating unit (a), the repeating number of the repeating unit (b1), and the repeating number of the repeating unit (b2) had a ratio of 4.5:1:1. In addition, the terminal group of the oligomer 20 was

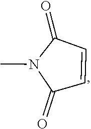

e.g. modified to contain a double bond,

### Example 1

First, 6 g of the oligomer 1, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of triallyl isocyanurate (TAIC) were then added into the solution and stirred to be completely dissolved, thereby obtaining a composition 1. The composition 1 was devolatilized and degassed, then processed to form a film at 150° C., and then cured at 200° C. for 3 hours to obtain a thermal cured product. The cured product had a dielectric constant (Dk@10 GHz) of 3.26, a dielectric loss (Df@10 GHz) of 0.0033, and a glass transition temperature of 208° C.

### Example 2

First, 6 g of the oligomer 2, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 2. The composition 2 was devolatilized and degassed, then processed to form a film at 150° C., and then cured at 200° C. for 3 hours to obtain a thermally cured product. The cured product had a dielectric constant (Dk@10 GHz) of 3.27, a dielectric loss (Df@10 GHz) of 0.0034, and a glass transition temperature of 210° C.

### Example 3

First, 6 g of the oligomer 3, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 3. The composition 3 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0033, and a glass transition temperature of 198° C.

### Example 4

First, 6 g of the oligomer 4, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 4. The composition 4 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0034, and a glass transition temperature of 207° C.

### Example 5

First, 6 g of the oligomer 5, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 5. The composition 5 was devolatilized and degassed, then processed to form a film at 150° C., and then cured at 200° C. for 3 hours to obtain a thermally cured product. The cured product had a dielectric constant (Dk@10 GHz) of 3.26, a dielectric loss (Df@10 GHz) of 0.0034, and a glass transition temperature of 209° C.

### Example 6

First, 6 g of the oligomer 6, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 6. The composition 6 was devolatilized and degassed, then processed to form a film at 150° C., and then cured at 200° C. for 3 hours to obtain a thermally cured product. The cured product had a dielectric constant (Dk@10 GHz) of 3.24, a dielectric loss (Df@10 GHz) of 0.0033, and a glass transition temperature of 207° C.

### Example 7

First, 6 g of the oligomer 7, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 7. The composition 7 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0034, and a glass transition temperature of 207° C.

### Example 8

First, 6 g of the oligomer 8, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 8. The composition 8 was devolatilized and degassed, then processed to form a film at 150° C., and then cured at 200° C. for 3 hours to obtain a thermally cured product. The cured product had a dielectric constant (Dk@10 GHz) of 3.26, a dielectric loss (Df@10 GHz) of 0.0035, and a glass transition temperature of 210° C.

### Example 9

First, 6 g of the oligomer 10, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 10. The composition 10 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0033, and a glass transition temperature of 206° C.

### Example 10

First, 6 g of the oligomer 12, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 12. The composition 12 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0033, and a glass transition temperature of 207° C.

### Example 11

First, 6 g of the oligomer 13, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 13. The composition 13 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0033, and a glass transition temperature of 197° C.

### Example 12

First, 6 g of the oligomer 15, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 15. The composition 15 was devolatilized and degassed, then processed to form a film at 150° C., and then cured at 200° C. for 3 hours to obtain a thermally cured product. The cured product had a dielectric constant (Dk@10 GHz) of 3.26, a dielectric loss (Df@10 GHz) of 0.0034, and a glass transition temperature of 211° C.

### Example 13

First, 6 g of the oligomer 16, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 16. The composition 16 was devolatilized and degassed, then processed to form a film at 150° C., and then cured at 200° C. for 3 hours to obtain a thermally cured product. The cured product had a dielectric constant (Dk@10 GHz) of 3.28, a dielectric loss (Df@10 GHz) of 0.0035, and a glass transition temperature of 213° C.

### Example 14

First, 6 g of the oligomer 19, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 19. The composition 19 was devolatilized and degassed, then processed to form a film at 150° C., and then cured at 200° C. for 3 hours to obtain a thermally cured product. The cured product had a dielectric constant (Dk@10 GHz) of 3.24, a dielectric loss (Df@10 GHz) of 0.0034, and a glass transition temperature of 212° C.

### Example 15

First, 6 g of the oligomer 20, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 20. The composition 20 was devolatilized and degassed, then processed to form a film at 150° C., and then cured at 200° C. for 3 hours to obtain a thermally cured product. The cured product had a dielectric constant (Dk@10 GHz) of 3.25, a dielectric loss (Df@10 GHz) of 0.0035, and a glass transition temperature of 214° C.

### Comparative Example 1

First, 6 g of the oligomer 9, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 9. The composition 9 was devolatilized and degassed, then processed to form a film at 150° C., and then cured at 200° C. for 3 hours to obtain a thermally cured product. The cured product had a dielectric constant (Dk@10 GHz) of 3.26, a dielectric loss (Df@10 GHz) of 0.0034, and a glass transition temperature of 196° C.

### Comparative Example 2

First, 6 g of the oligomer 11, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. 0.2 g of dicumyl peroxide serving as radical initiator and 8 g of TAIC were then added into the solution and stirred to be completely dissolved, thereby obtaining a composition 11. The composition 11 was devolatilized and degassed, then processed to form a film at 150° C., and then cured at 200° C. for 3 hours to obtain a thermally cured product. The cured product had a dielectric constant (Dk@10 GHz) of 3.27, a dielectric loss (Df@10 GHz) of 0.0043, and a glass transition temperature of 212° C.

### Comparative Example 3

First, 6 g of the oligomer 14, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. f0.2 g of dicumyl peroxide serving as radical initiator and 8 g of TAIC were then added into the solution and stirred to be completely dissolved, thereby obtaining a composition 14. The composition 14 was devolatilized and degassed, then processed to form a film at 150° C., and then cured at 200° C. for 3 hours to obtain a thermally cured product. The cured product had a dielectric constant (Dk@10 GHz) of 3.26, a dielectric loss (Df@10 GHz) of 0.0042, and a glass transition temperature of 210° C.

### Comparative Example 4

First, 6 g of the oligomer 17, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. f0.2 g of dicumyl peroxide serving as radical initiator and 8 g of TAIC were then added into the solution and stirred to be completely dissolved, thereby obtaining a composition 17. The composition 17 was devolatilized and degassed, then processed to form a film at 150° C., and then cured at 200° C. for 3 hours to obtain a thermally cured product. The cured product had a dielectric constant (Dk@10 GHz) of 3.27, a dielectric loss (Df@10 GHz) of 0.0043, and a glass transition temperature of 197° C.

### Comparative Example 5

First, 6 g of the oligomer 18, 14 g of polyphenylene ether oligomer SA9000 (commercially available from SABIC, having a methacryloxy terminal group), and 30 g of toluene were added into a reaction bottle, and then heated and stirred to be dissolved. After all the reactants were dissolved, the solution was cooled to room temperature. f0.2 g of dicumyl peroxide serving as radical initiator and 8 g of TAIC were then added into the solution and stirred to be completely dissolved, thereby obtaining a composition 18. The composition 18 was devolatilized and degassed, then processed to form a film at 150° C., and then cured at 200° C. for 3 hours to obtain a thermally cured product. The cured product had a dielectric constant (Dk@10 GHz) of 3.30, a dielectric loss (Df@10 GHz) of 0.0045, and a glass transition temperature of 200° C.

Accordingly, the low dielectric loss at high frequency (≤0.0035) and the high glass transition temperature (≥200° C.) could be simultaneously achieved in Examples of the disclosure. If no repeating unit (b1), (b2), or a combination thereof or the amount of repeating unit (b1), (b2), or a combination thereof was too low, the glass transition temperature would be insufficient. If the amount of repeating unit (a) was too low, the dielectric loss at high frequency would be too much. As shown in Comparative Examples 4 and 5, if the chemical structure of the repeating unit (b1) of the aliphatic cyclic diamine is different from the chemical structure of the repeating unit (b1) in the disclosure, the low dielectric loss at high frequency (≤0.0035) and the high glass transition temperature (≥200° C.) could not be simultaneously achieved.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. An oligomer, comprising:
a repeating unit 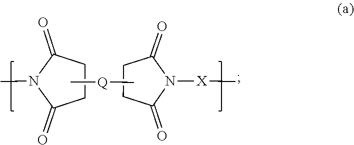
and
a repeating unit 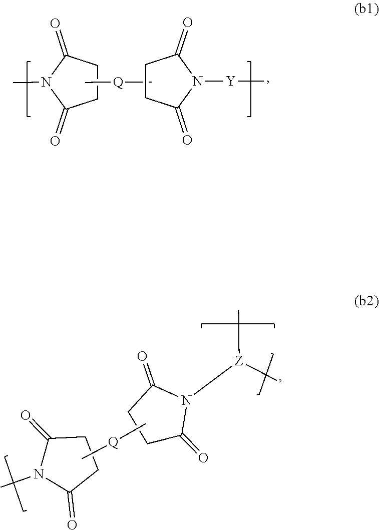
or a combination thereof,
wherein the repeating number of the repeating unit (a) and the repeating number of the repeating unit (b1), (b2), or a combination thereof have a ratio of 1:1 to 20:1;
each Q is independently substituted or unsubstituted aliphatic group, cycloaliphatic group, aromatic group, or siloxane group;
X is 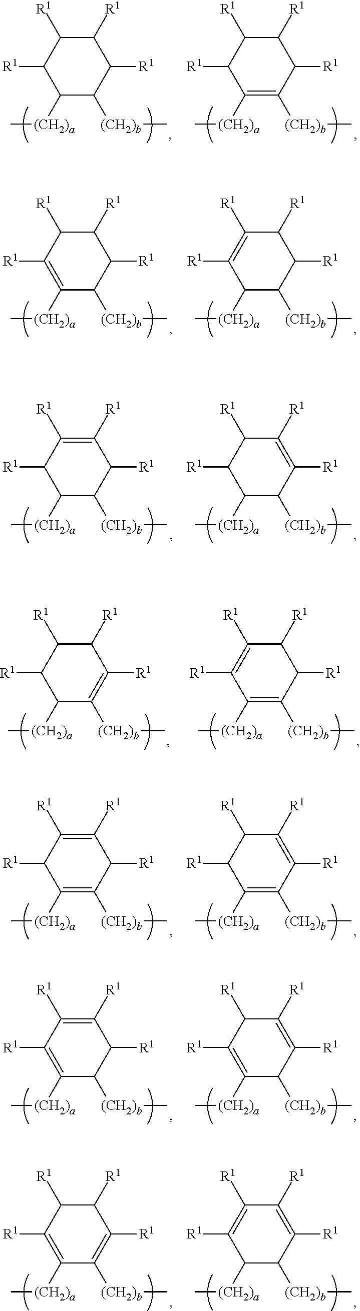 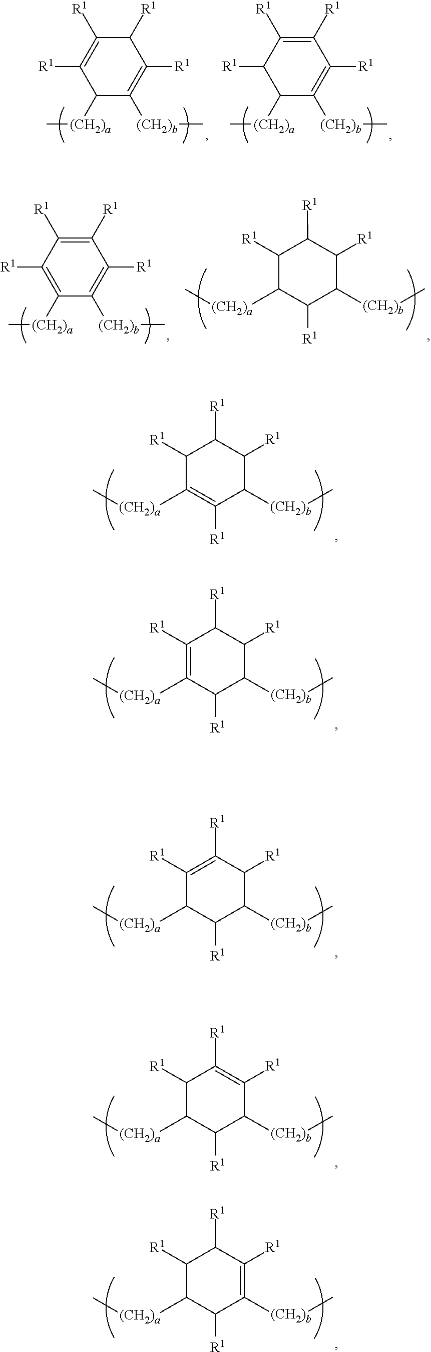 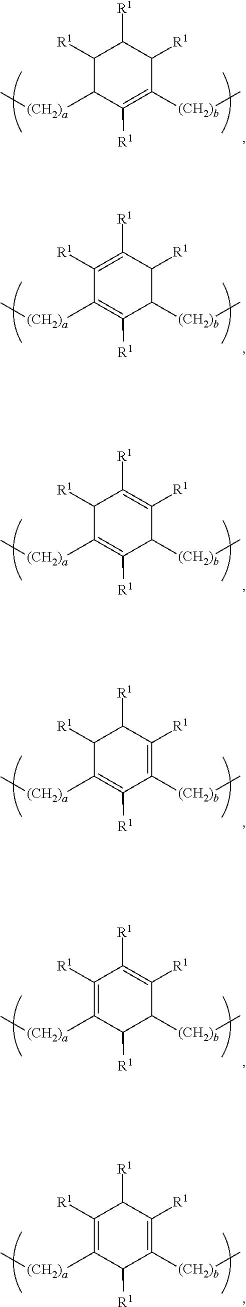 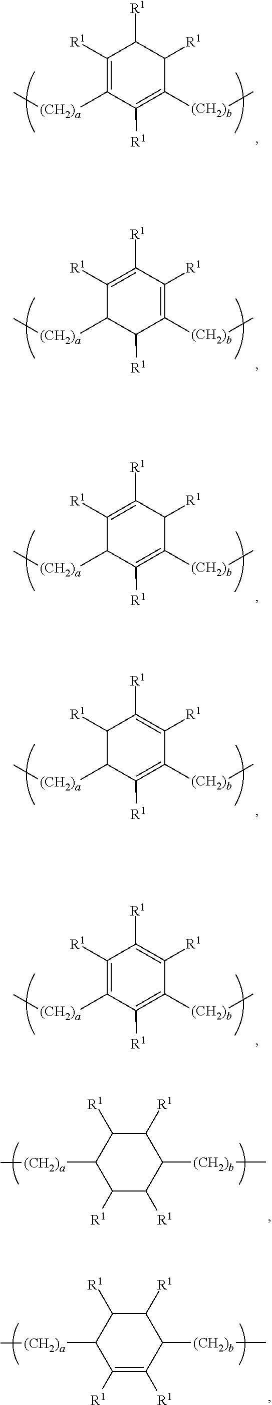 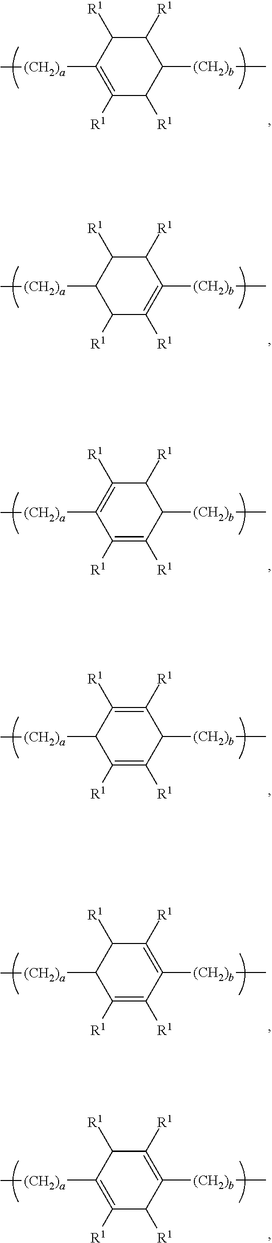 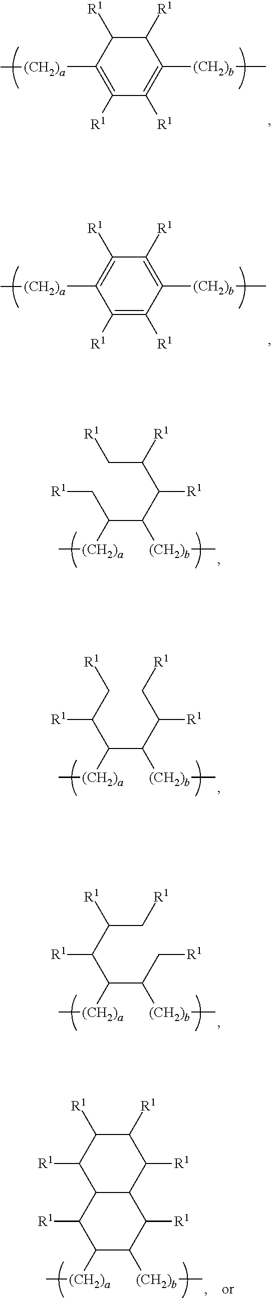 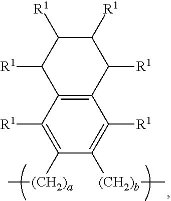
wherein 12≥a≥4; 12≥b≥4; each of R¹ is independently hydrogen, C₄₋₁₂ alkyl group, C₄₋₁₂ alkenyl group, or C₄₋₁₂ alkynyl group, and at least two R¹ is not hydrogen;
Y is 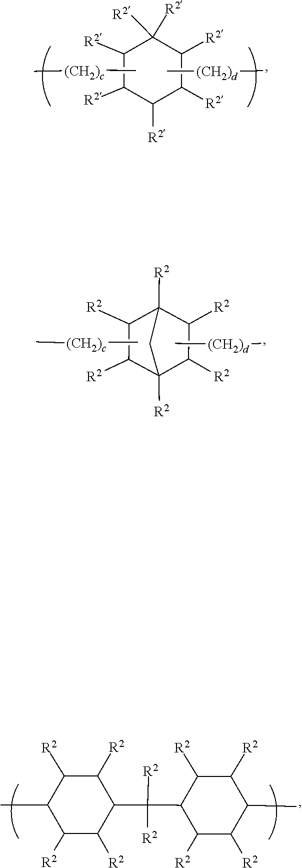
or a combination thereof, wherein c is 0 or 1; d is 0 or 1; each of R^{2′} is independently hydrogen or C₁₋₁₀ alkyl group, and at least one of R^{2′} is not hydrogen; and each of R² is independently hydrogen or C₁₋₁₀ alkyl group; and
Z is 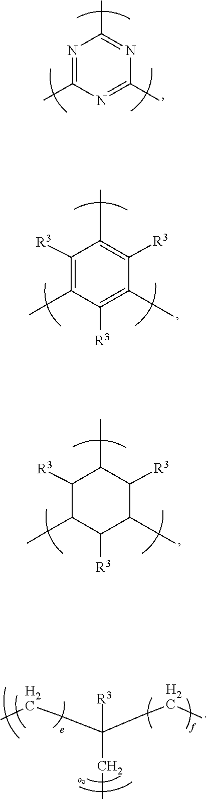
or a combination thereof, wherein 12≥e≥1; 12≥f≥1; 12≥g≥1; and each of R³ is independently hydrogen or C₁₋₁₀ alkyl group.

2. The oligomer as claimed in claim 1, wherein the oligomer comprises the repeating units (a), (b1), and (b2), and the repeating number of the repeating unit (b1) and the repeating number of the repeating unit (b2) have a ratio of 1:1 to 5:1.

3. The oligomer as claimed in claim 1, wherein the oligomer comprises the repeating units (a) and (b1), and the repeating number of the repeating unit (a) and the repeating number of the repeating unit (b1) have a ratio of 1:1 to 5:1.

4. The oligomer as claimed in claim 1, wherein 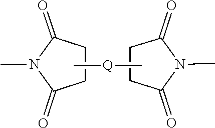
is 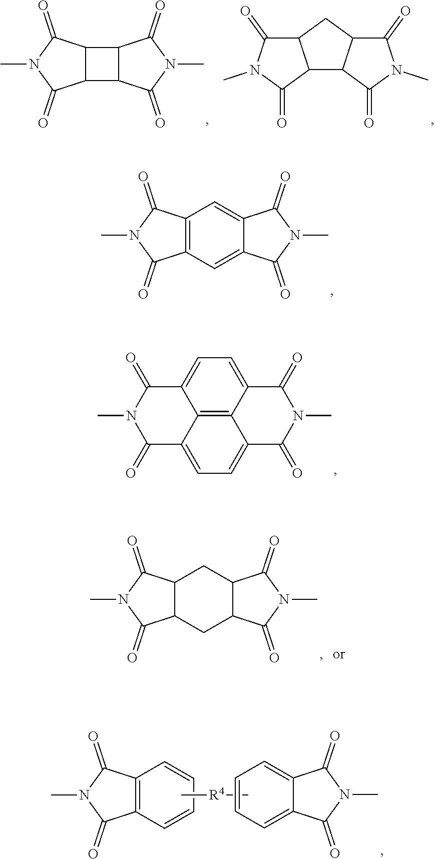
wherein R⁴ is C₁₋₅ alkylene group, —SO₂—, —O—, —C(═O)—, or a single bond.

5. The oligomer as claimed in claim 1, wherein the repeating unit (a) is 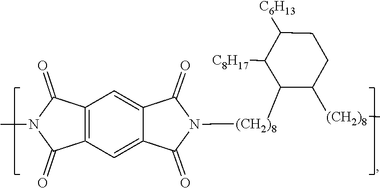
and the repeating unit (b1) is

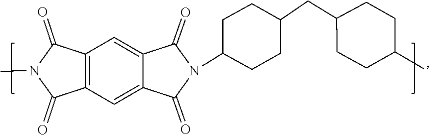

6. The oligomer as claimed in claim 1, wherein the repeating unit (a) is 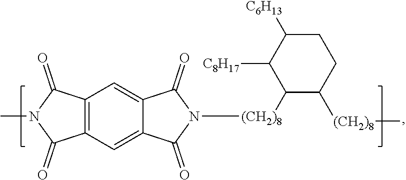
and the repeating unit (b1) is

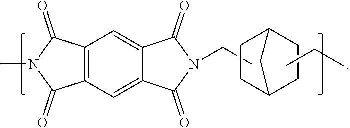

7. The oligomer as claimed in claim 1, wherein the repeating unit (a) is 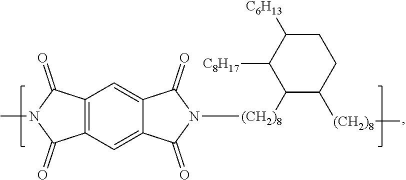
and the repeating unit (b1) is

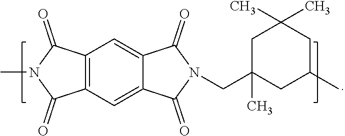

8. The oligomer as claimed in 1, wherein the repeating unit (a) is 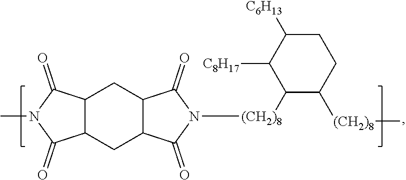
and the repeating unit (b1) is

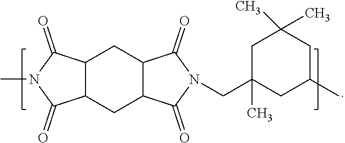

9. The oligomer as claimed in 1, wherein the repeating unit (a) is 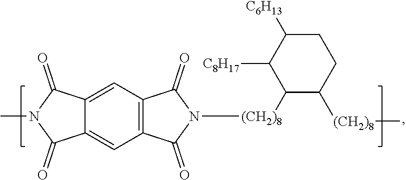
and the repeating unit (b2) is

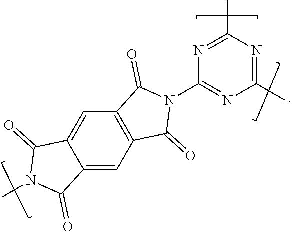

10. The oligomer as claimed in 1, wherein the repeating unit (a) is 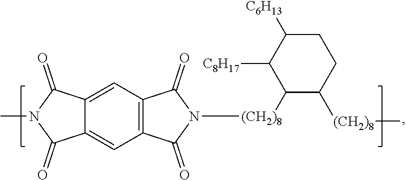
the repeating unit (b1) is 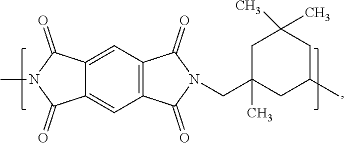
and the repeating unit (b2) is

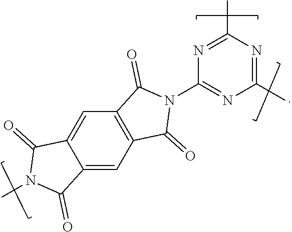

11. The oligomer as claimed in claim 1, having a terminal amino group or a terminal group modified to contain a double bond.

12. A composition, comprising:
an oligomer; and
a radical initiator,
wherein the oligomer has a terminal group modified to contain a double bond, and includes:
a repeating unit (a) 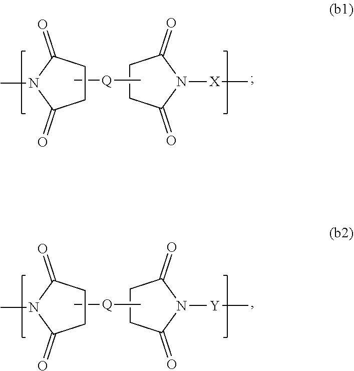
and
a repeating unit 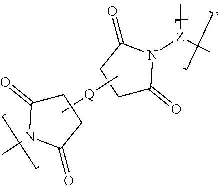
or a combination thereof,
wherein the repeating number of the repeating unit (a) and the repeating number of the repeating unit (b1), (b2) or a combination thereof have a ratio of 1:1 to 20:1;
each Q is independently substituted or unsubstituted aliphatic group, cycloaliphatic group, aromatic group, or siloxane group;
X is 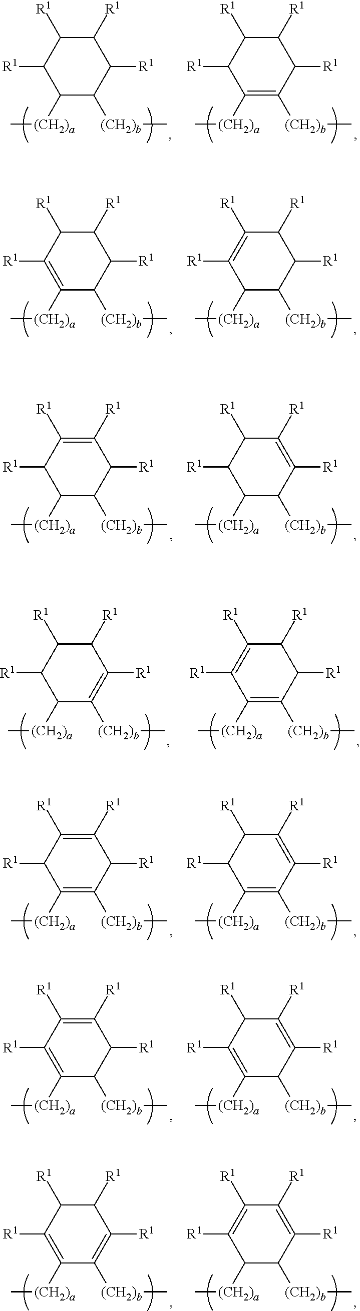 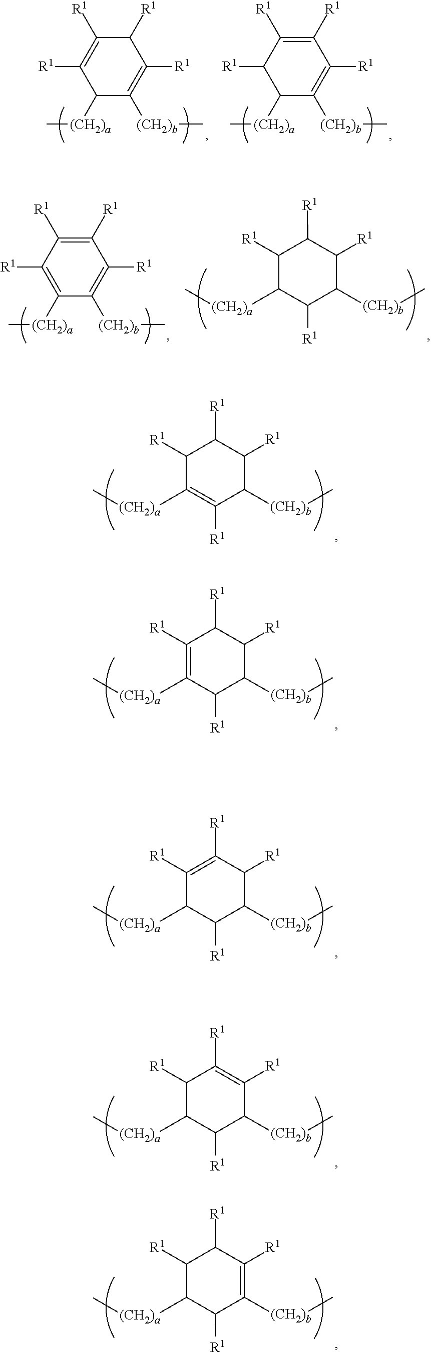 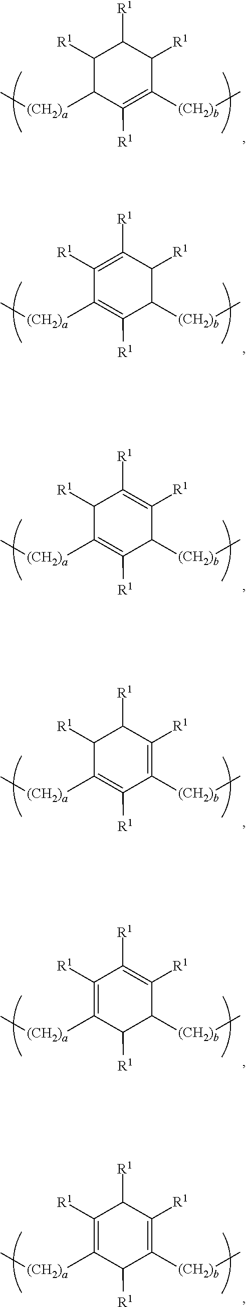 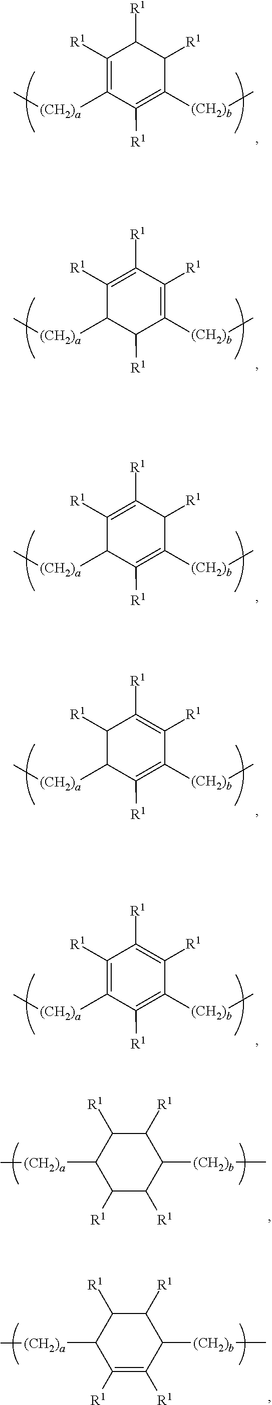 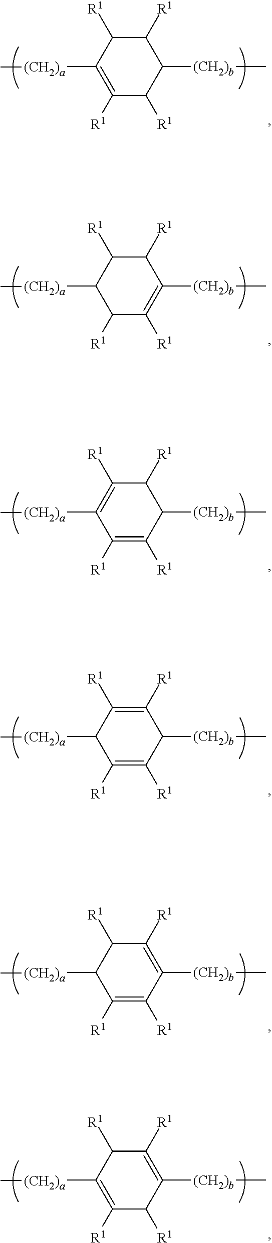 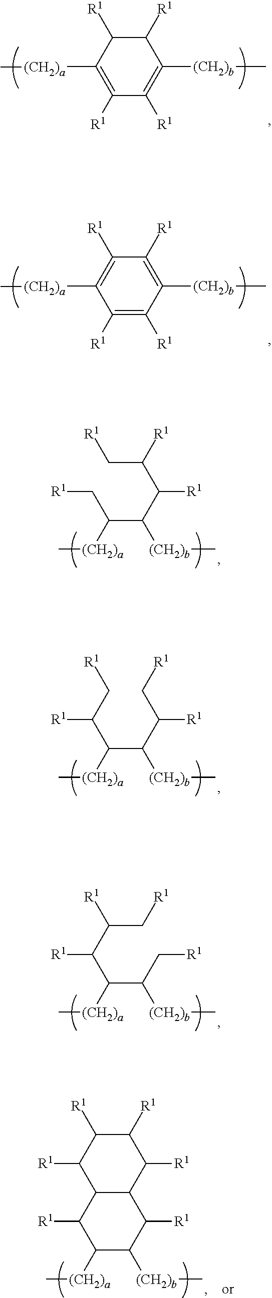 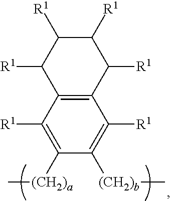
wherein 12≥a≥4; 12≥b≥4; each of R¹ is independently hydrogen, C₄₋₁₂ alkyl group, C₄₋₁₂ alkenyl group, or C₄₋₁₂ alkynyl group, and at least two R¹ is not hydrogen;
Y is 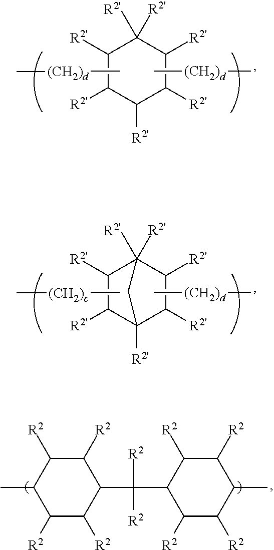
or a combination thereof, wherein c is 0 or 1; d is 0 or 1; each of R^{2′} is independently hydrogen or C₁₋₁₀ alkyl group, and at least one of R^{2′} is not hydrogen; and each of R² is independently hydrogen or C₁₋₁₀ alkyl group; and
Z is 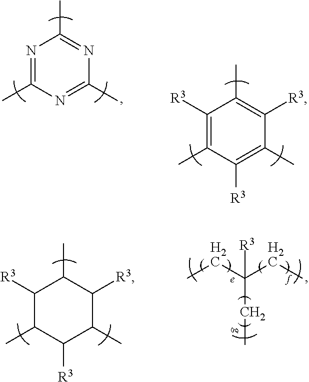
or a combination thereof, wherein 12≥e≥1; 12≥f≥1; 12≥g≥1; and each of R³ is independently hydrogen or C₁₋₁₀ alkyl group.

13. The composition as claimed in claim 12, further comprising a polyphenylene ether resin having a terminal methacryloxy group, a polyphenylene ether resin having a terminal vinyl benzyl ether group, or a combination thereof.
